(19) United States
(12) Patent Application Publication
(10) Pub. No.: US 2006/0083714 A1
(43) Pub. Date: Apr. 20, 2006

(54) Combination of a pde iv inhibitor and a tnf-alpha antagonist

(76) Inventor: James M Warner, Webster Groves, MO (US)

Correspondence Address: PHARMACIA CORPORATION;GLOBAL PATENT DEPARTMENT, POST OFFICE BOX 1027, ST. LOUIS, MO, 63006, US

(21) Appl. No.: 10/500,266

(22) PCT Filed: Jan. 23, 2004
(86) PCT No.: PCT/IB04/00616

§ 371 Date: Jun. 18, 2004

Related U.S. Application Data
(60) Provisional application No. 60/442,881, filed on Jan. 27, 2003.

(51) Int. Cl. A61K 39/395; A61K 38/16; A61K 38/21; A61K 31/5377; A61K 31/58; A61K 31/704; A61K 31/519; A61K 31/522

(52) U.S. Cl. 424/85.6; 514/220; 514/235.5; 514/262.1; 514/263.32; 514/406; 424/145.1; 514/454; 514/11; 514/6; 514/176; 514/323; 424/718; 424/725; 514/252.15; 514/266.3

(57) ABSTRACT

The subject invention relates to therapeutic combinations and methods for the treatment of inflammatory conditions and diseases. Particularly the present invention relates to treatments and methods for PDE IV-related conditions and for TNF-alpha-related conditions using a combination of a PDE IV inhibitor and a TNF-alpha antagonist.

### BACKGROUND OF THE INVENTION

1. Field of the Invention

This invention relates to therapeutic combinations and methods for the treatment of inflammatory conditions and diseases. Particularly the present invention relates to treatments and methods for PDE IV-related conditions and for TNF-alpha-related conditions.

2. Description of Related Art

Tumor necrosis factor-alpha (TNF-alpha) is a proinflammatory cytokine and plays a role in inflammatory and immunological events. The major sources of TNF-alpha are mast cells, eosinphils, macrophages, and monocytes. TNF-alpha causes a broad spectrum of effects both in vitro and in vivo, including vascular thrombosis and tumor necrosis, inflammation, activation of macrophages and neutrophils, leukocytosis, apoptosis, and shock. TNF-alpha has been associated with a variety of disease states including various forms of cancer, arthritis, psoriasis, endotoxic shock, sepsis, autoimmune diseases, infarctions, obesity, asthma, COPD, cachexia, stroke, glaucoma, retinitis, atherosclerosis and uveitis.

TNF-alpha activity can be reduced by treatment with, for example, an anti-TNF antibody. Examples of anti-TNF antibodies include, individually, etanercept or infliximab. An alternative therapy used to reduce TNF-alpha activity includes treating the patient with a glucocorticoid. Further individual therapies for the reduction of TNF-alpha activity are described by K. J. Tracey et al., Annu. Rev. Med. 45: 491-503 1994.

The enzyme phosphodiesterase-IV (PDE IV), is believed to be the predominant phosphodiesterase expressed within inflammatory cells. One of the primary activities of PDE IV is to metabolize excess intracellular levels of the signal transduction molecule cyclic adenosine 3′,5′-monophosphate (cAMP).

The molecule cAMP is a ubiquitous second messenger produced in cells in response to extracellular hormones and several neurotransmitters. The synthesis and release of proinflammatory mediators, cytokines (including TNF-alpha) and active oxygen species are inhibited where there is an increased level of cAMP (Dal Piaz, Eur. J. Med. Chem. 35: 463-480, 2000).

In contrast, native PDE IV activity causes reduction of intracellular cAMP and is associated with triggering the release of several inflammatory cellular mediators including histamine and several cytokines, thus resulting in the symptoms of inflammation. Chemical inhibition of PDE IV activity has been found to increase intracellular levels of cAMP, which in turn, down-regulate the harmful activity of inflammatory cells.

Multiple isoforms of the phosphodiesterase enzyme have been identified that differ in their substrate specificity, kinetic properties, responsiveness to endogenous regulators (Ca2+/calmodulin, cyclic GMP), and susceptibility to inhibition by various compounds. Phosphodiesterase isoforms include the phosphodiesterases 1-10. For purposes of the present invention, the preferred PDE isoform to be inhibited, is the cAMP-specific type-4 PDE (PDE IV). Within the category of the PDE IV isoform, there are 4 known subtypes. The PDE IV subtypes, A through D, are all specific for cyclic AMP, but differ in terms of their mRNA splicing and upstream conserved domains. However, all 4 subtypes, A-D, are included within the scope of the term, “PDE IV”, for purposes of the present invention.

PDE inhibitors like theophylline and pentoxyphylline inhibit all or most PDE isozymes indiscriminately in all tissue. These compounds exhibit side effects, apparently because they nonselectively inhibit multiple PDE isozyme classes in a variety of tissues. The target disease may be effectively treated by such compounds, but unwanted secondary side effects may be exhibited which, if they could be avoided or minimized, would increase the overall therapeutic effect of this approach to treating certain diseases. See PCT publication WO 01/60358 A1. Examples of compounds that inhibit multiple isoforms, in addition to PDE IV, of the PDE enzyme include theophylline, quinazolines, ibudilast, benafentrine zardaverine, and pentoxyfyllin.

The therapeutic use a of PDE IV inhibitor with a PDE III inhibitor is described in PCT publication number WO 00/66123. A method of treatment using a PDE IV inhibitor and a corticosteroid is described in PCT publication number WO 01/32127 A2.

Asthma affects about 10 million Americans, about a third of whom are under 18 years of age. In the United States alone billions of dollars are spent annually on asthma-related health care. The episodic breathing difficulty that characterizes asthma is brought about by a combination of three primary factors including 1) bronchospasm, i.e. variable and reversible airway obstruction due to airway muscle contraction, 2) inflammation of the airway lining, and 3) bronchial hyper-responsiveness that results in excessive mucus in the airways. Triggers of asthma attacks vary among individuals, but common triggers include allergens such as dust mites and mold, environmental pollutants, viral agents, and physical exertion or exercise.

The Mayo Clinic reports that chronic obstructive pulmonary disease (COPD), mostly emphysema or chronic bronchitis, kills 85,000 people a year in the United States. Chronic obstructive pulmonary disease actually refers collectively to several chronic or progressive pulmonary diseases including asthmatic bronchitis, chronic bronchitis (with normal airflow), chronic obstructive bronchitis, bullous disease, and emphysema, all involving inflammation. For example, chronic bronchitis involves an inflammation and eventual scarring of the lining of the bronchial tubes producing symptoms including chronic cough, increase of mucus, frequent clearing of the throat and shortness of breath. Emphysema results from the normal but chronic inflammatory response of the airway lining to chronic exposure to environmental pollutants such as cigarette smoke.

Drug treatment for asthma and COPD includes intravenous, oral, subcutaneous or inhaled administration of bronchodilators including beta-adrenergics, methyl xanthines, and anti-cholinergics, and also administration of corticosteroids, the mast cell mediator-release inhibitors known as Cromolyn and Tilade, or, more recently, anti-leukotrienes, for anti-inflammatory effects. However, the cellular and molecular mechanisms of inflammatory and immune processes that play a role in the pathogenesis and progression of asthma and COPD are not yet well understood.

### SUMMARY OF THE INVENTION

Briefly, therefore, the present invention is directed to a method for the treatment or prophylaxis of a PDE IV- or a TNF-alpha-related condition in a mammal in need of such treatment or prophylaxis, comprising administrating to the mammal an amount of a PDE IV inhibitor and an amount of a TNF-alpha antagonist wherein the amount of the PDE IV inhibitor and the amount of the TNF-alpha antagonist together comprise an effective therapy for the treatment or prevention of a PDE IV- or a TNF-alpha-related condition.

The invention is further directed to a therapeutic composition comprising an amount of a PDE IV inhibitor and an amount of a TNF-alpha antagonist and a pharmaceutically acceptable excipient.

Another embodiment of the present invention provides a kit for the purpose of treatment or prophylaxis of a PDE IV- or a TNF-alpha-related condition in a mammal in need of such treatment or prophylaxis, the kit comprising a dosage form comprising a PDE IV inhibitor and a dosage form comprising a TNF-alpha antagonist.

Further scope of the applicability of the present invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DETAILED DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

a. Definitions

The following definitions are provided in order to aid the reader in understanding the detailed description of the present invention:

The term “asthma” refers to a respiratory disorder characterized by episodic difficulty in breathing brought on by any one or a combination of three primary factors including: 1) bronchospasm, i.e. variable and reversible airway obstruction due to airway muscle contraction, 2) inflammation of the airway lining, and 3) bronchial hyper-responsiveness resulting in excessive mucus in the airways, which may be triggered by exposure to an allergen or combination of allergens such as dust mites and mold, viral or bacterial infection especially infection with a “common cold” virus, environmental pollutants such as chemical fumes or smoke, physical over exertion such as during exercise, stress, or inhalation of cold air. The terms “chronic obstructive pulmonary disease” and “COPD” as used interchangeably herein refers to a chronic disorder or combination of disorders characterised by, for example, reduced maximal expiratory flow and slow forced emptying of the lungs that does not change markedly over several months and is not, or is only minimally, reversible with traditional bronchodilators. Commonly, COPD involves a combination of chronic bronchitis, i.e. the presence of cough and sputum for more than three months for about two consecutive years, and emphysema, i.e. alveolar damage. However, COPD can involve singly or in combination chronic bronchitis with normal airflow, chronic bronchitis with airway obstruction (chronic obstructive bronchitis), emphysema, asthmatic bronchitis, or bullous disease.

The term “respiratory disease or condition” refers to any one of several ailments that involve inflammation and affect a component of the respiratory system including especially the trachea, bronchi and lungs. Such ailments can include without limitation asthmatic conditions such as allergen-induced asthma, exercise-induced asthma, pollution-induced asthma, cold-induced asthma, stress-induced asthma and viral-induced-asthma, chronic obstructive pulmonary diseases including chronic bronchitis with normal airflow, chronic bronchitis with airway obstruction (chronic obstructive bronchitis), emphysema, asthmatic bronchitis, or bullous disease. The term “respiratory disease or condition” can also include without limitation other pulmonary diseases involving inflammation including cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin-protamine reactions, sepsis, status asthmaticus and hypoxia.

The terms “phosphodiestrease inhibitor” and “PDE inhibitor” as used interchangeably herein denote a compound that reduces the physiological effect of a phosphodisterase enzyme, for example slowing the degradation of cyclic AMP (cAMP) or cyclic (cGMP).

The term “PDE IV inhibitor” denotes a compound that is capable of reducing the in vitro enzyme activity of the PDE IV isoform of phosphodiesterase.

A PDE IV inhibitor may show different in vitro IC₅₀ values with respect to different isoforms of PDE. The in vitro IC₅₀ value exhibited by a compound for the inhibition of another isoform of PDE (herein, “PDE Z) divided by the IC₅₀ value for the inhibition of PDE IVis referred to herein as “inter-isoform selectivity” with respect to that other PDE isoform.

The term “inter-isoform selective PDE IV inhibitor” refers to a PDE IV inhibitor for which its inter-isoform selectivity with respect to another PDE isoform is greater than one.

It is believed that there are at least two binding forms on human monocyte recombinant PDE IV (human PDE IV) at which inhibitors bind. One explanation for these observations is that human PDE IV exists in two distinct forms. One binds rolipram with high affinity while the other binds rolipram with low affinity. Herein we distinguish these forms by referring to them as the high affmity rolipram binding form (HPDE IV) and the low affinity binding form (LPDE IV). It has been reported that certain compounds which potently compete for HPDE IV have more side effects or more intense side effects than those which more potently compete with LPDE IV (see, for example, U.S. Pat. No. 5,998,428, herein incorporated by reference). Further data indicate that compounds can be targeted to the low affinity binding form of PDE IV and that this form is distinct from the binding form for which rolipram is a high affmity binder. Compounds that interact with LPDE IV are reported to have anti-inflammatory activity, whereas those that interact with the HPDE IV produce side effects or exhibit more intensely those side effects. Rolipram binds to one catalytic site of one form with a high affinity (HPDE IV), defined herein as having a Kᵢ less than 10 nanomolar, and to the other form with a low affinity (LPDE IV), defined here as having a Kᵢ of greater than 100 nanomolar. U.S. Pat. No. 5,998,428 describes a method of measuring the in vitro IC₅₀ ratios for a compound with respect to HPDE IV and LPDE IV.

As used herein, the term “intra-isoform selectivity” with respect to a particular compound refers to its in vitro lC₅₀ with respect to HPDE IV divided by its in vitro IC₅₀ with respect to LPDE IV.

The term “intra-isoform selective PDE IV inhibitor” means a PDE IV inhibitor for which the intra-isoform selectivity is about 0.1 or greater.

The terms “selective phosphodiesterase IV inhibitor” and “selective PDE IV inhibitor” denote a compound which exhibits either an inter-isoform selective PDE IV inhibitor or an intra-isoform selective PDE IV inhibitor.

The term “subject” as used herein refers to an animal, in one embodiment a mammal, and in an exemplary embodiment particularly a human being, who is the object of treatment, observation or experiment. In another embodiment the mammal can be, for example, a companion animal such as a dog, a cat, or a horse.

The terms “dosing” and “treatment” as used herein refer to any process, action, application, therapy or the like, wherein a subject, particularly a human being, is rendered medical aid with the object of improving the subject's condition, either directly or indirectly.

The term “therapeutic compound” as used herein refers to a compound useful in the prophylaxis or treatment of a disease or condition.

The term “therapeutically effective” as used herein refers to a characteristic of an amount of a therapeutic compound, or a characteristic of amounts of combined therapeutic compounds in combination therapy. The amount or combined amounts achieve the goal of preventing, avoiding, reducing or eliminating the respiratory disease or condition.

“Combination therapy” means the administration of two or more therapeutic agents to treat a condition. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating.the condition.

The term “pharmaceutically-acceptable salt” embraces salts commonly used to form alkali metal salts and to form addition salts of free acids or free bases. The nature of the salt is not critical, provided that it is pharmaceutically acceptable or compatible with a medical therapy. Pharmaceutically acceptable salts are particularly useful as products of the methods of the present invention because of their greater aqueous solubility relative to a corresponding parent or neutral compound. Such salts must have a pharmaceutically acceptable anion or cation. Suitable pharmaceutically acceptable acid addition salts of compounds of the present invention may be prepared from inorganic acid or from an organic acid. Examples of such inorganic acids are hydrochloric, hydrobromic, hydroiodic, nitric, carbonic, sulfuric and phosphoric acid. Appropriate organic acids include from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, or galacturonic acid. Suitable pharmaceutically-acceptable base addition salts of compounds of the present invention include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procaine. Suitable pharmnaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic (including carbonate and hydrogen carbonate anions), sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. The chloride salt is particularly preferred for medical purposes. Suitable pharmaceutically acceptable base salts include ammonium salts, alkali metal salts such as sodium and potassium salts, and alkaline earth salts such as magnesium and calcium salts. All of these salts may be prepared by conventional means from the corresponding conjugate base or conjugate acid of the compounds useful in the present invention by reacting, respectively, the appropriate acid or base with the conjugate base or conjugate acid of the compound.

b. Detailed Description

In accordance with the present invention, there is now provided a method for the treatment or prophylaxis of a PDE IV- or a TNF-alpha-related condition in a mammal in need of such treatment or prophylaxis comprising administrating to the mammal an amount of a PDE IV inhibitor and an amount of a TNF-alpha antagonist wherein the amount of the PDE IV inhibitor and the amount of the TNF-alpha antagonist together comprise an effective therapy for the treatment or prevention of a PDE IV- or a TNF-alpha-related condition. Preferably the PDE IV inhibitor is a selective PDE IV inhibitor.

For purposes of the present invention, the terms “PDE IV inhibitor” refer to any compound that is known to inhibit the PDE IV enzyme or which is discovered to act as a PDE IV inhibitor (PDE IV antagonist). PDE IV inhibitors include any compound that is known or can be discovered to inhibit the PDE IV enzyme regardless of whether the compound also demonstrates inhibition of other isoforms of the phosphodiesterase enzyme (PDE).

It is preferred that the PDE IV inhibitor that is used in the present invention is one that is a PDE IV selective inhibitor.

To determine the inter-isoform selectivity of a PDE IV inhibitor, the putative inhibitor compound is typically incubated together with each individual isoform of phosphodiesterase and simultaneously with substrate cyclic nucleotides. PDE inhibition is then determined by the presence or absence of substrate degradation products. See e.g. Hatzelmann, A., et al., J. Pharm. Exper. Therap., 297(1):267-279 (2001). The relative ability of an inhibitory compound to slow or prevent the degradation of tritiated cyclic nucleotides is one test that is indicative of how well the compound in question selects one or more of each isoform to inhibit. Representative PDE isoform enzymes and other reaction substrates can be obtained by isolation from appropriate tissues and their purchase has been reported.

In practice, the in vitro selectivity of a PDE IV inhibitor may vary depending upon the condition under which the test is performed and on the inhibitors being tested. However, for the purposes of this specification, the selectivity of a PDE IV inhibitor can be measured as a ratio of the in vitro IC₅₀ value for inhibition of any other isoform of the phosphodiesterase enzyme (Z) other than PDE IV, divided by the IC₅₀ value for inhibition of PDE IV (PDE Z IC₅₀/PDE IV IC₅₀), where Z identifies any PDE other than PDE IV. As used herein, the term “IC₅₀” refers to the concentration of a compound that is required to produce 50% inhibition of phosphodiesterase activity. A PDE IV selective inhibitor is any inhibitor for which the ratio of PDE Z IC₅₀to PDE IV IC₅₀ is greater than 1. In a preferred embodiment, this ratio is greater than 2, more preferably greater than 10, yet more preferably greater than 100, and more preferably still greater than 1000.

By way of example, in Hatzelmann, A., et al., J. Pharm. Exper. Therap., 297(1) 267-279 (2001), the IC₅₀ for roflumilast activity on PDE IV was reported to be 0.0008 μM, while the IC₅₀ for roflumilast activity on PDE I was reported to be >10 μM. Accordingly, the selectivity of roflumilast for PDE IV as compared with PDE I would be >10/0.0008 or at least about 12,500. Likewise, the selectivity of roflumilast for PDE IV as compared with PDE V would be 8/0.0008 or at least about 10,000.

Thus, preferred PDE IV selective inhibitors of the present invention have a PDE IV IC₅₀ of less than about 1 μM, more preferred of less than about 0.1 μM, even more preferred of less than about 0.01 μM, and more preferred still of less than about 0.001 μM. Preferred PDE IV selective inhibitors have a PDEZ IC₅₀ of greater than about 1 μM, and more preferably of greater than 10 μM.

An example of a selective PDE IV inhibitor that is particularly preferred for use in the present invention has been recently described for use in treating pulmonary inflammation is the pyridyl benzamide derivative, roflumilast (3-cyclopropylmethloxy-4-difluoromnethoxy-N-[3,5-dichloropyrid-4-yl]-benzamide), a novel, highly potent, and selective PDE4 inhibitor. See U.S. Pat. No. 5,712,298, which in herein incorporated by reference.

PDE IV inhibitors are classified into three main chemical classes 1) Catechol Ethers (in which are grouped a wide variety of flexible molecules of inhibitors structurally related to rolipram) 2) Quinazolinediones which are structurally related to Nitraquazone and 3) Xanthines, to which theophylline belongs. Inside this class, two subclasses can be distinguished quinazolindiones and xanthines.

Preferably the PDE IV inhibitor is selected from the group consisting of rolipram, roflumilast, cilomilast, and ZK-117137, bamifylline, dyphylline, ibudilast, and Theophylline. Further individual PDE IV inhibitors useful in the present invention are individually listed in Table 1.

TABLE 1

| No. | Structure I.D. | Structure | Structure Name | Reference |
|---|---|---|---|---|
| 1. | cilomilast Ariflo SB- 207499 CAS RN: 153259- 65-5 | 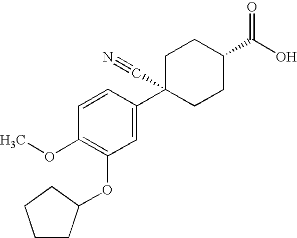 | 4-cyano-4-[3- cyclopentyloxy)-4- methoxy phenyl]cyclohexane carboxylic acid | Dal Piaz, V., et. al., Eur. J. Med. Chem. 35 (2000) 463-480 |
| 2. | roflumilast BY-217 CAS RN: 162401-32-3 | 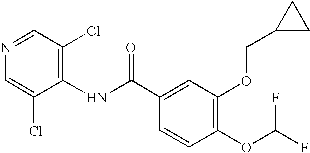 | 3-(cyclopropylmethoxy)- N-(3,5-dichloropyridin- 4-yl)-4- (difluoromethoxy) benzamide | Souness, J., et al., Immunopharmacology 47 (2000) 127-162 |
| 3. | Pumafentrin BYK-33043 BY-343 CAS RN: 207993-12-2 | 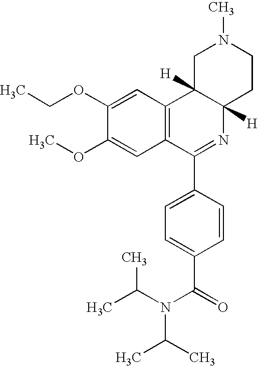 | 4-(9-Ethoxy-8-methoxy- 2-methyl- 1,2,3,4,4a,10b- hexahydro-benzo [c][1,6] napthyridin-6- yl)-N,N-di isopropyl- benzamide | Norman P., Expert Opin. Ther. Patents (2002) 12(1):93-111 |
| 4. | L-869298 CT-2450 Analogue: CT-2820 CT- 3883 L-826141 Analogue: L- 791943 CT-5210 CAS RN: 225919-29-9 | 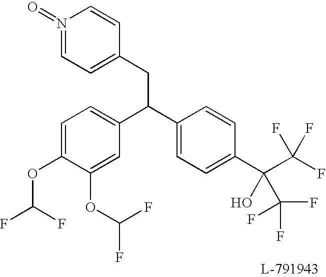 | 2-{4-[1-[3,4- bis(difluoromethoxy) phenyl]-2-(1- oxidopyridin-4- yl)ethyl]phenyl]- 1,1,1,3,3,3- hexafluoropropan-2-ol | Norman P., Expert Opin. Ther. Patents (2002) 12(1):93-111 |
| 5. | ZK-117137 SH-636 Trade Name: Mesopram CAS RN: 189940-24-7 | 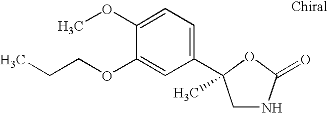 | 5-(4-methoxy-3- propoxyphenyl)-5- methyl-1,3-oxazolidin- 2-one | US 2002/010310 6 A1 |
| 6. | rolipram ME- 3167 ZK- 62711 CAS RN: 61413-54-5 | 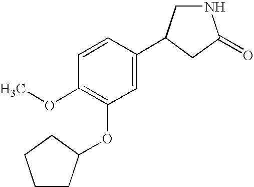 | 4-(3-cyclopentyloxy- 4-methoxy-phenyl)- pyrrolidan-2-one | Dal Piaz, V., et. al., Eur. J. Med. Chem. 35 (2000) 463-480 |
| 7. | YM-976 CAS RN: 191219- 80-4 | 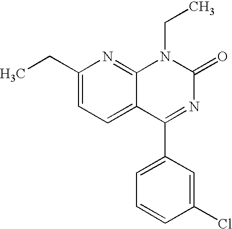 | 4-(3-Chloro-phenyl)-1,7- diethyl-1H-pyrido[2,3- d]pyrimidin-2-one | US 2002/010310 6 A1 |
| 8. | Sch-351591 D-4396 | 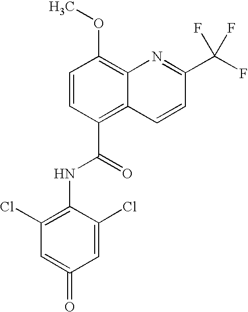 | N-(3,5-dichloro-1- oxidopyridin-4-yl)-8- methoxy-2- (trifluoromethyl)quinoline- 5-carboxamide | US 2002/010310 6 A1 |
| 9. | IC-485 | 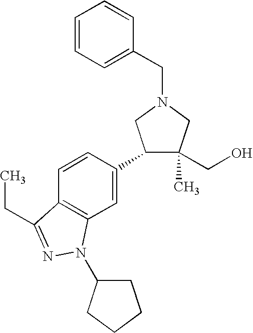 | [1-benzyl-4-(1- cyclopentyl-3-ethyl-1H- indazol-6-yl)-3- methylpyrrolidin-3- yl]methanol | US 2002/010310 6 A1 |
| 10. | D-4418 Sch- 365351 CAS RN: 257892- 34-5 | 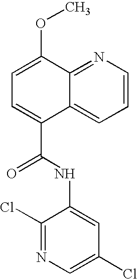 | 8-methoxy-quinoline-5- carboxylic acid (2,5- dichloropyridin-3-yl) amide | US 2002/010310 6 A1 |
| 11. | PD-189659 CI-1044 Analogue: PD-168787 CI-1018 Analogue: PD-190749 Analogue: PD-190036 CAS RN: 197894-84-1 (Pfizer) | 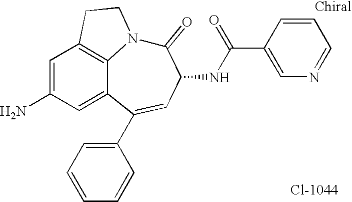 | N-[9-amino-4-oxo-7- phenyl-1,2,4,5- tetrahydroazepino[3,2,1- hi]indol-5- yl]nicotinamide | Dal Piaz, V., et. al., Eur. J. Med. Chem. 35 (2000) 463-480 |
| 12. | CP-77059 CAS RN: 114918-24-0 | 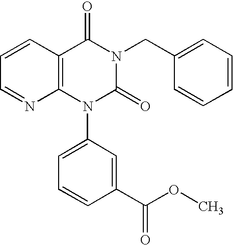 | 3-(3-benzyl-2,4-dioxo- 3,4-dihydro-2H- pyrido[2,3-d]pyrimidin- 1-yl)benzoic acid methyl ester | Dal Piaz, V., et. al., Eur. J. Med. Chem. 35 (2000) 463-480 |
| 13. | RS-14203 CAS RN: 150347-75-4 | 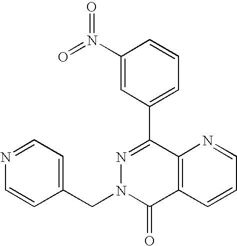 | 8-(3-nitrophenyl)-6- (pyridin-4-ylmethyl) pyrido[2,3-d] pyridazin- 5(6H)-one | Dal Piaz, V., et. al., Eur. J. Med. Chem. 35 (2000) 463-480 |
| 14. | AWD-12-281 Analogue: AWD-12-343 CAS RN: 257892-33-4 | 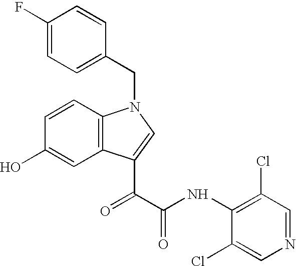 | N-(3,5-dichloropyridin- 4-yl)-2-[1-(4- fluorobenzyl)-5- hydroxy-1H-indol-3-yl]- 2-oxoacetamide | US 2002/010310 6 A1 |
| 15. | D-22888 Analogue: AWD-12-232 CAS RN: 182282-60-6 | 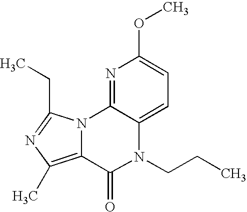 | 9-ethyl-2-methoxy-7- methyl-5- propylimidazo[1,5- a]pyrido[3,2-e]pyrazin- 6(5H)-one | Dal Piaz, V., et. al., Eur. J. Med. Chem. 35 (2000) 463-480 |
| 16. | YM-58977 | 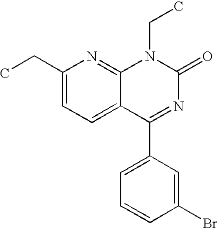 | 4-(3-bromophenyl)-1,7- diethylpyrido[2,3- d]pyrimidin-2(1H)-one | Dal Piaz, V., et. al., Eur. J. Med. Chem. 35 (2000) 463-480 |
| 17. | Theophylline CAS RN: 58-55-9 | 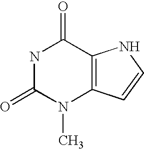 | 3,7-Dihydro-1,3- dimethyl-1H-purine-2,6- dione | Dal Piaz, V., et. al., Eur. J. Med. Chem. 35 (2000) 463-480 |
| 18. | Cipamfylline HEP-688 BRL-61063 CAS RN: 132210-43-6 | 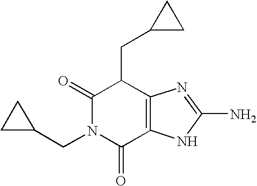 | 8-amino-1,3-bis- cyclopropylmethyl-3,7- dihydro-purine-2,6- dione | Dal Piaz, V., et. al., Eur. J. Med. Chem. 35 (2000) 463-480 |
| 19. | Arofylline LAS-31025 CAS RN: 136145-07-8 | 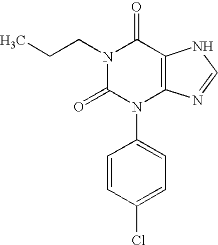 | 3-(4-chlorophenyl)-1- propyl-3,7-dihydro-1H- purine-2,6-dione | Dal Piaz, V., et. al., Eur. J. Med. Chem. 35 (2000) 463-480 |
| 20. | V-11294A CAS RN: 162278-09-3 | 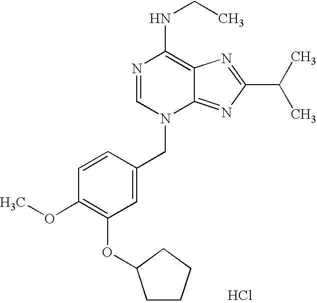 | [3-(3-cyclopentyloxy-4- methoxybenzyl]-8-8- isopropyl-3H-purin-6-yl]- ethyl amine hydrochloride | Dal Piaz, V., et. al., Eur. J. Med. Chem. 35 (2000) 463-480 |
| 21. | RPR-132294 Analogue: RPR-132703 | 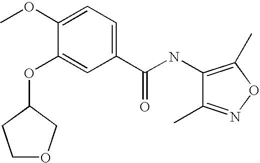 | N-(3,5- dimethylisoxazol-4-yl)- 4-methoxy-3- (tetrahydrofuran-3- yloxy)benzamide | Dal Piaz, V., et. al., Eur. J. Med. Chem. 35 (2000) 463-480 |
| 22. | IBMX CAS RN: 28822- 58-4 | 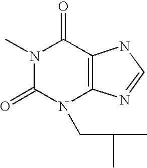 | 3-isobutyl-1-methyl-3,7- dihydro-1H-purine-2,6- dione | Dal Piaz, V., et. al., Eur. J. Med. Chem. 35 (2000) 463-480 |
| 23. | Isbufylline CAS RN: 90162-60-0 | 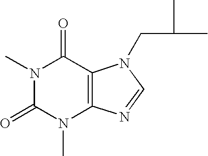 | 7-isobutyl-1,3-dimethyl- 3,7-dihydro-1H-purine- 2,6-dione | Dal Piaz, V., et. al., Eur. J. Med. Chem. 35 (2000) 463-480 |
| 24. | Doxofylline Trade Names: Ansimar Maxivent CAS RN: 69975-86-6 | 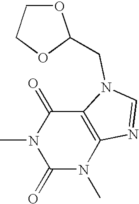 | 7-(1,3-dioxolan-2- ylmethyl)-1,3-dimethyl- 3,7-dihydro-1H-purine- 2,6-dione | Dal Piaz, V., et. al., Eur. J. Med. Chem. 35 (2000) 463-480 |
| 25. | Dyphylline CAS RN: 479-18-5 | 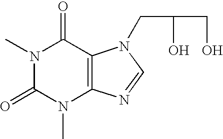 | 7-(2,3-dihydroxypropyl)- 1,3-dimethyl-3,7- dihydro-1H-purine-2,6- dione | Dal Piaz, V., et. al., Eur. J. Med. Chem. 35 (2000) 463-480 |
| 26. | Verofylline CAS RN: 65029-11-0 | 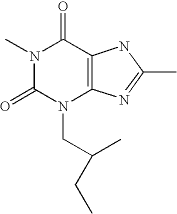 | 1,8-dimethyl-3-(2- methylbutyl)-3,7- dihydro-1H-purine-2,6- dione | Dal Piaz, V., et. al., Eur. J. Med. Chem. 35 (2000) 463-480 |
| 27. | Bamifylline CAS RN: 2016-63-9 | 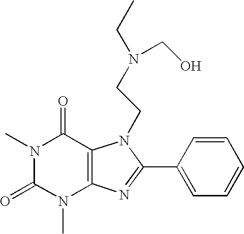 | 7-{2-[ethyl(hydroxy- methyl)amino]ethyl]-1,3- dimethyl-8-phenyl-3,7- dihydro-1H-purine-2,6- dione | Dal Piaz, V., et. al., Eur. J. Med. Chem. 35 (2000) 463-480 |
| 28. | Pentoxifylline CAS RN: 6493-05-6 | 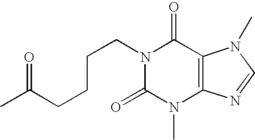 | 3,7-dimethyl-1-(5- oxohexyl)-3,7-dihydro- 1H-purine-2,6-dione | Dal Piaz, V., et. al., Eur. J. Med. Chem. 35 (2000) 463-480 |
| 29. | Enprofylline CAS RN: 41078-02-8 | 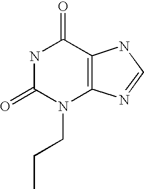 | 3-propyl-3,7-dihydro- 1H-purine-2,6-dione | Dal Piaz, V., et. al., Eur. J. Med. Chem. 35 (2000) 463-480 |
| 30. | Denbufylline CAS RN: 57076-71-8 | 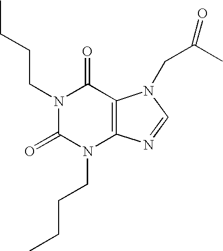 | 1,3-dibutyl-7-(2- oxopropyl)-3,7-dihydro- 1H-purine-2,6-dione | Dal Piaz, V., et. al., Eur. J. Med. Chem. 35 (2000) 463-480 |
| 31. | Chiroscience 245412 | 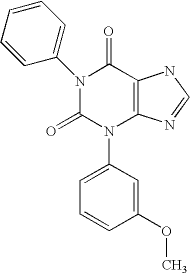 | 3-(3-methoxyphenyl)-1- phenyl-3,7-dihydro-1H- purine-2,6-dione | Dal Piaz, V., et. al., Eur. J. Med. Chem. 35 (2000) 463-480 |
| 32. | ICI 63197 CAS RN: 27277-00-5 | 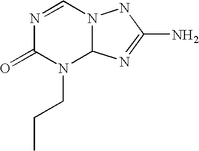 | 2-amino-4-propyl-3a,4- dihydro[1,2,4]triazolo[1,5- a][1,3,5]triazin-5(1H)- one | Dal Piaz, V., et. al., Eur. J. Med. Chem. 35 (2000) 463-480 |
| 33. | SCA 40 | 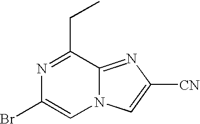 | 6-bromo-8- ethylimidazo[1,2- a]pyrazin-2-amine | Dal Piaz, V., et. al., Eur. J. Med. Chem. 35 (2000) 463-480 |
| 34. | Ibudilast CAS RN: 50847-11-5 | 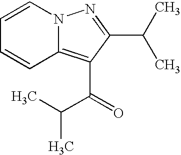 | 1-(2-isopropyl- pyrazolo[1,5- a]pyridin-3-yl)-2- methylpropan-1-one | Dal Piaz, V., et. al., Eur. J. Med. Chem. 35 (2000) 463-480 |
| 35. | N-cyclopentyl- 8-cyclopropyl- 3-propyl-3H- purin-6-amine CAS RN: 162278-16-2 162278-06-0 | 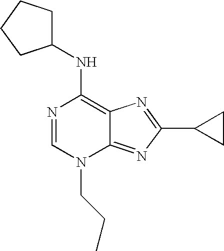 | N-cyclopentyl-8- cyclopropyl-3-propyl- 3H-purin-6-amine | Dal Piaz, V., et. al., Eur. J. Med. Chem. 35 (2000) 463-480 |
| 36. | 8-cyclopropyl- N,3-diethyl-3H- purin-6-amine CAS RN: 126149-38-0 126252-48-0 126371-20-0 | 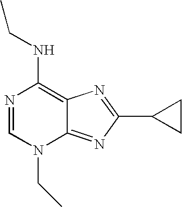 | 8-cyclopropyl-N,3- diethyl-3H-purin-6- amine | Dal Piaz, V., et. al., Eur. J. Med. Chem. 35 (2000) 463-480 |
| 37. | INN: lirimilast BAY-19-8004 CAS RN: 329306-27-6 | 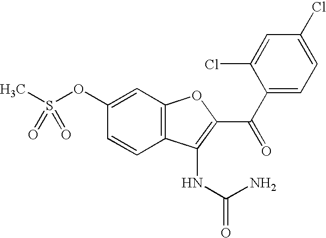 | Methane suifonic acid 2-(2,4-dichloro-benzoyl- 3-ureido-benzofuran-6- yl ester | Dal Piaz, V., et. al., Eur. J. Med. Chem. 35 (2000) 463-480 |
| 38. | (4-chlorophenyl)- [3-(3,3- dihydroxybutyl)- 6-hydroxy-1- benzofuran-2- yl]methanone | 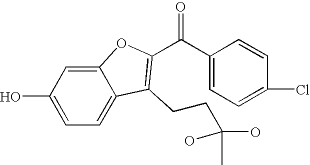 | (4-chlorophenyl)[3-(3,3- dihydroxybutyl)-6- hydroxy-1-benzofuran- 2-yl]methanone | Dal Piaz, V., et. al., Eur. J. Med. Chem. 35 (2000) 463-480 |
| 39. | 1-{3- (dimethylamino)- 4-[(dimethylamino)- methyl]-7-hydroxy- 5,6-dimethyl-1- benzofuran-2- yl}ethanone | 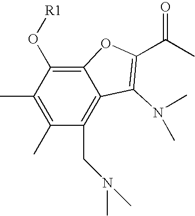 | 1-{3-(dimethylamino)-4- [(dimethylamino)methyl]- 7-hydroxy-5,6- dimethyl-1-benzofuran- 2-yl}ethanone | Dal Piaz, V., et. al., Eur. J. Med. Chem. 35 (2000) 463-480 |
| 40. | N-(3,5- dichloropyridin-4- yl)-8-methoxy-2,2- dimethylchromane- 5-carboxamide | 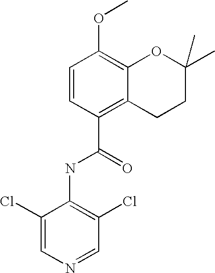 | N-(3,5-dichloropyridin- 4-yl)-8-methoxy-2,2- dimethylchromane-5- carboxamide | Dal Piaz, V., et. al., Eur. J. Med. Chem. 35 (2000) 463-480 |
| 41. | 2-acetyl-N- benzyl-7- methoxy-1- benzofuran-4- sulfonamide | 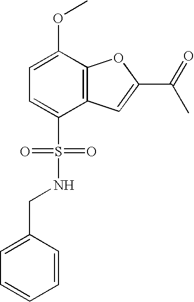 | 2-acetyl-N-benzyl-7- methoxy-1-benzofuran- 4-sulfonamide | Dal Piaz, V., et. al., Eur. J. Med. Chem. 35 (2000) 463-480 |
| 42. | 1-cyclopentyl- N-(3,5-dichloro pyridin-4-yl)-3- ethyl-1H-indazole- 6-carboxamide | 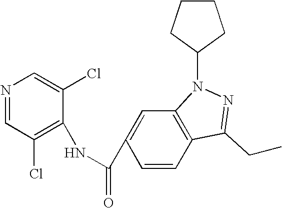 | 1-cyclopentyl-N-(3,5- dichloropyridin-4-yl)-3- ethyl-1H-indazole-6- carboxamide | Dal Piaz, V., et. al., Eur. J. Med. Chem. 35 (2000) 463-480 |
| 43. | 1-cyclopentyl- 3-ethyl-6-(2- methylphenyl)- 1,3a,4,5,6,7a- hexahydro-7H- pyrazolo[3,4- c]pyridin-7-one | 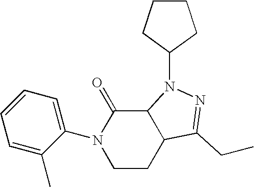 | 1-cyclopentyl-3-ethyl-6- (2-methylphenyl)- 1,3a,4,5,6,7a- hexahydro-7H- pyrazolo[3,4-c]pyridin- one | Dal Piaz, V., et. al., Eur. J. Med. Chem. 35 (2000) 463-480 |
| 44. | N-(4-oxo-1- phenyl-3,4,6,7- tetrahydro[1,4]diazepino[6,7,1- hi]indol-3-yl)- 1H-indole-2- carboxamide | 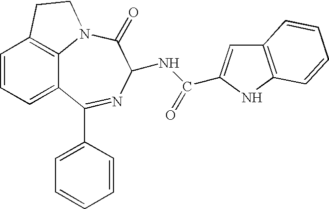 | N-(4-oxo-1-phenyl- 3,4,6,7- tetrahydro[1,4]diazepino [6,7,1-hi]indol-3-yl)-1H- indole-2-carboxamide | Dal Piaz, V., et. al., Eur. J. Med. Chem. 35 (2000) 463-480 |
| 45. | CI-1118 | 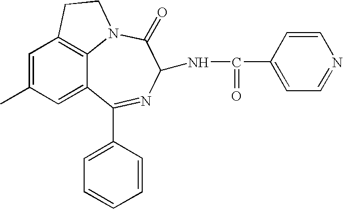 | N-(9-methyl-4-oxo-1- phenyl-3,4,6,7- tetrahydro[1,4]diazepino [6,7,1-hi]indol-3- yl)isonicotinamide | Dal Piaz, V., et. al., Eur. J. Med. Chem. 35 (2000) 463-480 |
| 46. | 4-[4-(cyclopropyl-6- (cyclopropylamino)- 1,3,5-triazin-2-yl]- 1lambda˜4˜,4- thiazinane-1,1- diol | 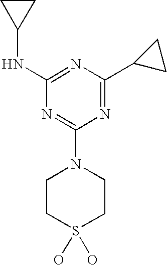 | 4-[4-cyclopropyl-6- (cyclopropylamino)- 1,3,5-triazin-2-yl]- 1lambda˜4˜,4- thiazinane-1,1-diol | Dal Piaz, V., et. al., Eur. J. Med. Chem. 35 (2000) 463-480 |
| 47. | N-cyclopropyl-4-(2- methylcyclopropyl)- 6-(2-methyl- morpholin- 4-yl)-1,3,5- triazin-2-amine | 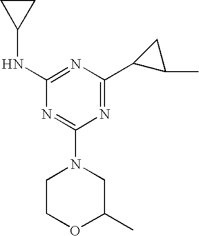 | N-cyclopropyl-4-(2- methylcyclopropyl)-6-(2- methylmorpholin-4-yl)- 1,3,5-triazin-2-amine | Dal Piaz, V., et. al., Eur. J. Med. Chem. 35 (2000) 463-480 |
| 48. | Atizoram CP 80633 CAS RN: 135637-46-6 | 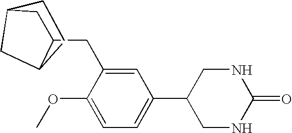 | 2(1H)-Pyrimidinone, 5- [3-[(1S,2S,4R)- bicyclo[2.2.1]hept-2- yloxy]-4-methoxyphenyl]- tetrahydro- | Souness, J., et al., Immunopharmacology 47 (2000) 127-162 |
| 49. | Filaminast WAY-PDA-641 CAS RN: 141184-34-1 | 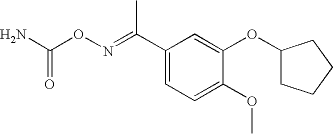 | Ethanone, 1-(3- (cyclopentyloxy)-4- methoxyphenyl)-,O- (aminocarbonyl) oxime, (E) | Souness, J., et al., Immunopharmacology 47 (2000) 127-162 |
| 50. | Piclamilast RP 73401 RPR 73401 CAS RN: 144035-83-6 | 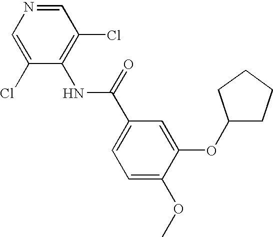 | Benzamide, 3- (cyclopentyloxy)-N-(3,5- dichloro-4-pyridinyl)-4- methoxy | Dal Piaz, V., et. al., Eur. J. Med. Chem. 35 (2000) 463-480 |
| 51. | Tibenelast Sodium LY 186655 CAS RN: 105102-18-9 | 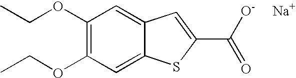 | Sodium 5,6- diethoxybenzo(b)-thioph ene-2-carboxylate | Souness, J., et al., Immunopharmacology 47 (2000) 127-162 |
| 52. | CDP 840 CAS RN: 162542-90-7 | 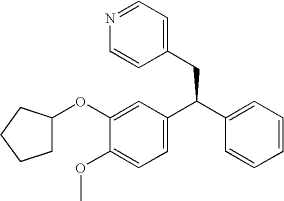 | Pyridine, 4-[(2R)-2-[3- (cyclopentyloxy)-4- methoxyphenyl]-2- phenylethyl]- | Souness, J., et al., Immunopharmacology 47 (2000) 127-162 |
| 53. | GW 3600 GL 193600X CAS RN: 173258-94-1 | 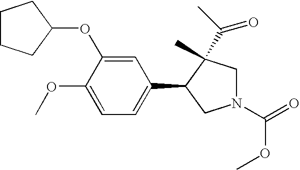 | 1-Pyrrolidinecarboxylic acid, 3-acetyl-4-[3- (cyclopentyloxy)-4- methoxyphenyl]-3- methyl-, methyl ester, (3R,4R) | US 2002/010310 6 A1 |
| 54. | NCS 613 CAS RN: 190377-71-0 | 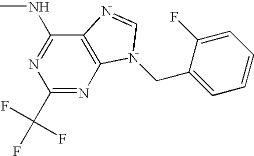 | 9H-Purin-6-amine, 9- [(2-fluorophenyl)methyl]- N-methyl-2- (trifluoromethyl)- | US 2002/010310 6 A1 |
| 55. | PDB 093 | No Structure | | US 2002/010310 |
| | CAS RN: | | | 6 A1 |
| | 444657-05-0 | | | |
| 56. | Ro 20-172 CAS RN: 29925-17-5 | 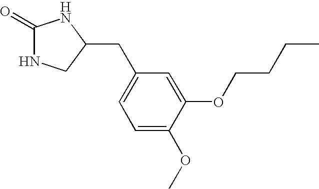 | 2-Imidazolidinone, 4- [(3-butoxy-4- methoxyphenyl)methyl] | US 2002/010310 6 A1 |
| 57. | RS 25344- 000 CAS RN: 152814-89-6 | 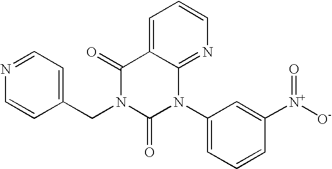 | Pyrido[2,3-d]pyrimidine- 2,4(1H,3H)-dione, 1-(3- nitrophenyl)-3-(4- pyridinylmethyl) | Dal Piaz, V., et. al., Eur. J. Med. Chem. 35 (2000) 463-480 |
| 58. | SKF 107806 | No Structure | | US 2002/010310 |
| | CAS RN: | | | 6 A1 |
| | 444615-76-3 | | | |
| 59. | XT-44 CAS RN: 135462-05-4 | 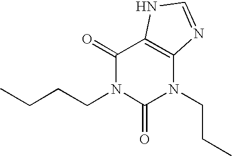 | 1-n-butyl-3-n- propylxanthine | Waki, Y., et al., Jpn J Pharmacol 79(4): 477-83 (1999) |
| 60. | tolafentrine | 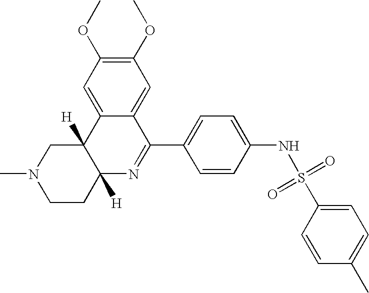 | Benzenesulfonamide, N-[4-[(4aR,10bS)- 1,2,3,4,4a,10b-hexahydro- 8,9-dimethoxy-2- methylbenzo[c][1,6]- naphthyridin-6-yl]phenyl]- 4-methyl | US 2002/010310 6 A1 |
| 61. | zardaverine | 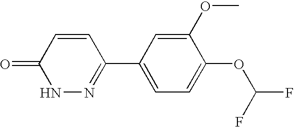 | 3(2H)-Pyridazinone, 6- [4-(difluoromethoxy)-3- methoxyphenyl] | Souness, J., et al., Immunopharmacology 47 (2000) 127-162 |
| 62. | T-2585 | 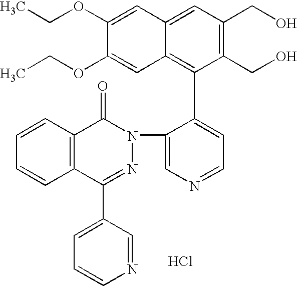 | 2-[4-(6,7-Diethoxy-2,3- bis-hydroxymethyl- napthalen-1-yl)-pyridin- 3-yl]-4-pyridin-3-yl-2H- phthalazin-1-one; compound with generic inorganic neutral component | US 2002/010310 6 A1 |
| 63. | SDZ-ISQ- 844 | 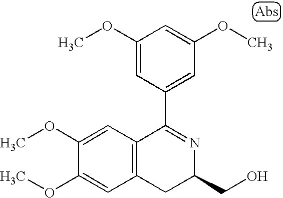 | [1-(3,5-Dimethoxy- phenyl)-6,7-dimethoxy- 3,4-dihydro-isoquinolin- 3-yl]-methanol | US 2002/010310 6 A1 |
| 64. | SB 207499 | 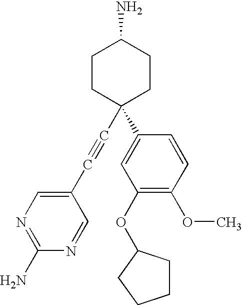 | 5-[4-Amino-1-(3- cyclopentyloxy-4- methoxy-phenyl)- cyclohexylethynyl]- pyrimidin-2-ylamine | Souness, J., et al., Immunopharmacology 47 (2000) 127-162 |
| 65. | RPR- 117658A | 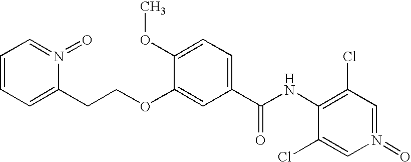 | N-(3,5-Dichloro-1-oxy- pyridin-4-yl)-4-methoxy- 3-[2-(1-oxy-pyridin-2-yl)- ethoxy]-benzamide | US 2002/010310 6 A1 |
| 66. | L-787258 | No structure | | US 2002/010310 |
| | | | | 6 A1 |
| 67. | E-4021 | 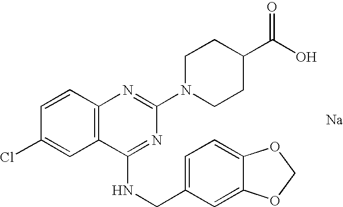 | 1-{4-[(Benzo[1,3]dioxol- 5-ylmethyl)-amino]-6- chloro-quinazolin-2-yl}- piperidine-4-carboxylic acid; compound with generic inorganic neutral component | US 2002/010310 6 A1 |
| 68. | GF-248 | 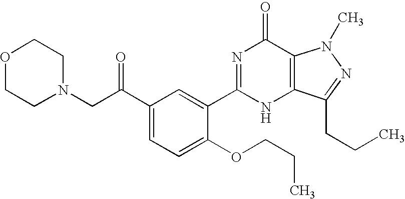 | 1-Methyl-5-[5-(2- morpholin-4-yl-axetyl)- 2-propoxy-phenyl]-3- propyl-1,4-dihydro- pyrazolo[4,3- d]pyrimidin-7-one | US 2002/010310 6 A1 |
| 69. | IPL-4088 | No structure | | US 2002/010310 |
| | | | | 6 A1 |
| 70. | CP-353164 | 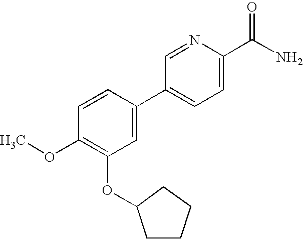 | 5-(3-Cyclopentyloxy-4- methoxy-phenyl)- pyridine-2-carboxylic acid amide | US 2002/010310 6 A1 |
| 71. | CP-146523 | 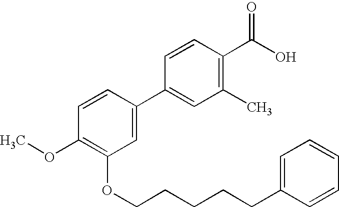 | 4′-Methoxy-3-methyl-3′- (5-phenyl-pentyloxy)- biphenyl-4-carboxylic acid | US 2002/010310 6 A1 |
| 72. | CP-293321 | No structure | | US 2002/010310 |
| | | | | 6 A1 |
| 73. | XT-611 | 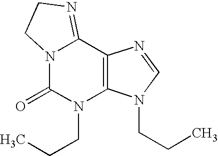 | 3,4-Dipropyl-3,4,6,7- tetrahydro-1,3,4,5a,8- pentaaza-as-indacen-5-one | US 2002/010310 6 A1 |
| 74. | WAY- | No structure | | US 2002/010310 |
| | 126120 | | | 6 A1 |
| 75. | WAY- 122331 | 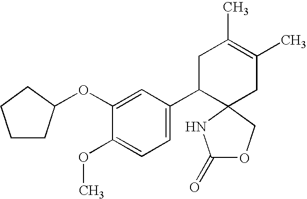 | 1-(3-Cyclopentoxy-4- methoxy-phenyl)-7,8- dimethyl-3-oxa-1-aza- spiro[4.5]dec-7-en-2-one | US 2002/010310 6 A1 |
| 76. | WAY- 127093B | 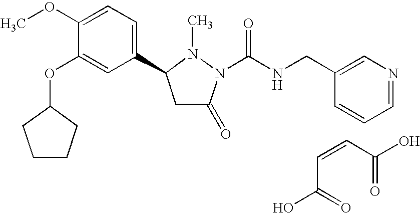 | 3-(3-Cyclopentyloxy-4- methoxy-phenyl)-2- methyl-5-oxo- pyrazolidine-1- carboxylic acid (pyridin- 3-ylmethyl)-amide; compound with but-2- enedioic acid | US 2002/010310 6 A1 |
| 77. | PDB-093 | No structure | | US 2002/010310 |
| | | | | 6 A1 |
| 78. | CDC-801 | 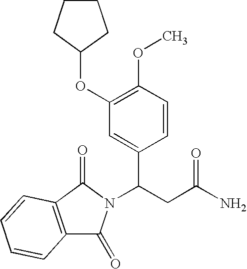 | 3-(3-Cyclopentyloxy-4- methoxy-phenyl)-3-(1.3- dioxo-1,3-dihydro- isoindol-2-yl)- propionamide | US 2002/010310 6 A1 |
| 79. | CC-7085 | No structure | | US 2002/010310 |
| | | | | 6 A1 |
| 80. | CDC-998 | No structure | | US 2002/010310 |
| | | | | 6 A1 |
| 81. | CH-3697 | No structure | | US 2002/010310 |
| | | | | 6 A1 |
| 82. | CH-3442 | No structure | | US 2002/010310 |
| | | | | 6 A1 |
| 83. | CH-2874 | No structure | | US 2002/010310 |
| | | | | 6 A1 |
| 84. | CH-4139 | No structure | | US 2002/010310 |
| | | | | 6 A1 |
| 85. | RPR- 114597 | 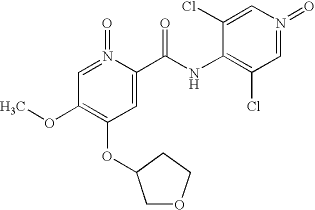 | 5-Methoxy-1-oxy-4- (tetrahydro-furan-3- yloxy)-pyridine-2- carboxylic acid (3,5- dicloro-1-oxy-pyridin-4- yl) amide | US 2002/010310 6 A1 |
| 86. | RPR- 122818 | 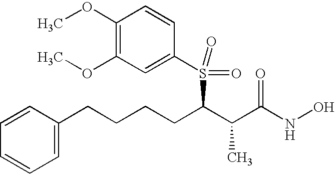 | 3-3(3,4-Dimethoxy- bemzenesulfonyl)-2- methyl-7-phenyl- heptanoic acid hydroxamide | US 2002/010310 6 A1 |
| 87. | KF-19514 | 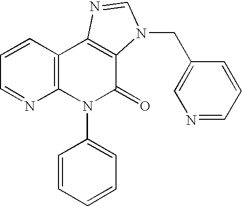 | 5-Phenyl-3-pyridin-3- ylmethyl-3,5-dihydro- 1,3,5,6-tetraaza- cyclopenta[a]- naphthalene-A-one | US 2002/010310 6 A1 |
| 88. | CH-422 | No structure | | US 2002/010310 |
| | | | | 6 A1 |
| 89. | CH-673 | No structure | | US 2002/010310 |
| | | | | 6 A1 |
| 90. | CH-928 | No structure | | US 2002/010310 |
| | | | | 6 A1 |
| 91. | KW-4490 | No structure | | US 2002/010310 |
| | | | | 6 A1 |
| 92. | Org 20241 | 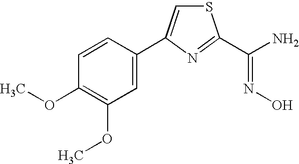 | 4-(3,4-Dimethoxy- phenyl)-N-hydroxy- thiazole-2- carboxamidine | US 2002/010310 6 A1 |
| 93. | Org 30029 | 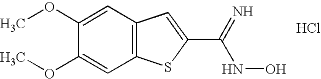 | N-Hydroxy-5,6- dimethoxy-benzo[b]- thiopene-2carboxamidine; compound with a generic inorganic neutral component | US 2002/010310 6 A1 |
| 94. | VMX 554 | No Structure | | New Drugs for |
| | VMX 565 | | | Respiratory |
| | | | | Diseases, 5^{th} |
| | | | | International |
| | | | | Conference, |
| | | | | San Diego, CA, |
| | | | | USA, July 3-5, 2002 |
| 95. | Benafentrine | 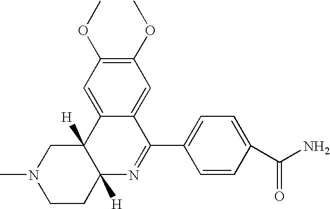 | Acetamide, N-[4- [(4aR,10bS)- 1,2,3,4,4a,10b-hexahydro- 8,9-dimethoxy-2- methylbenzo[c][1,6]- napthyridin-6-yl]phenyl] | US 6,333,354 B1 |
| 96. | Trequinsin | 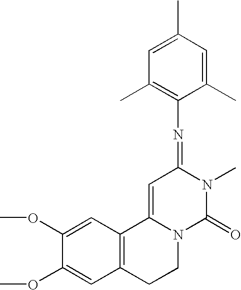 | 4H-Pyrimido[6,1- a]isoquinolin-4-one, 2,3,6,7-tetrahydro-9,10- dimethoxy-3-methyl-2- [(2,4,6-trimethyl- phenyl)imino] | US 6,333,354 B1 |
| 97. | EMD 54622 | 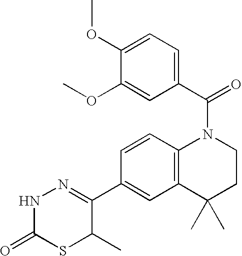 | Quinoline, 6-(3,6- dihydro-6-methyl-2-oxo- 2H-1,3,4-thiadiazin-5-yl)- 1-(3,4-dimethoxybenzoyl)- 1,2,3,4-tetrahydro-4,4- dimethyl | US 6,333,354 B1 |
| 98. | RS 17597 | 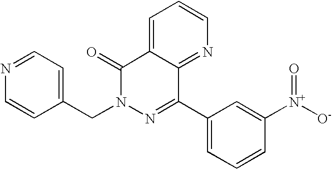 | Pyrido[2,3-d]pyridazin- 5(6H)-one, 8-(3- nitrophenyl)-6-(4- pyridinylmethyl) | US 2002/010310 6 A1 |
| 99. | Nitraquazone | 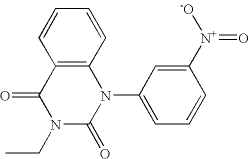 | 2,4(1H,3H)- Quinazolinedione, 3- ethyl-1-(3-nitrophenyl) | Dal Piaz, V., et. al., Eur. J. Med. Chem. 35 (2000) 463-480 |
| 100. | Oxagrelate | 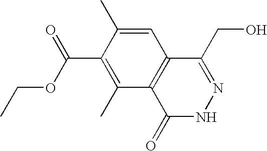 | 6-Phthalazinecarboxylic acid, 3,4-dihydro-1- (hydroxymethyl)-5,7- dimethyl-4-oxo-, ethyl ester | US 6,333,354 B1 |

In one embodiment the PDE IV inhibitor is a catechol ether selected from the group consisting of cilomilast, roflumilast, pumafentrin, L-869298, ZK-117137, and rolipram. In a preferred embodiment the PDE IV inhibitor is cilomilast. In another preferred embodiment the PDE IV inhibitor is roflumilast. In another preferred embodiment the PDE IV inhibitor is rolipram.

In another embodiment the PDE IV inhibitor is a quinazolidione or related compound selected from the group consisting of YM-976, Sch-351591, IC-485, Sch-365351, PD-189659, CP-77059, RS-14203 e, AWD-12-281, D-22888, and YM-58977.

In another embodiment the PDE IV inhibitor is a xanthine or related compound selected from the group consisting of Theophylline, cipamfylline, arofylline, V-11294A, RPR-132294, IBMX, isbufylline, doxofylline, dyphylline, verofylline, bamifylline, pentoxiylline, enprofylline, denbufylline, Chiroscience 245412, ICI-63197, SCA-40, ibudilast, N-cyclopentyl-8-cyclopropyl-3-propyl-3H-purin-6-amine, and 8-cyclopropyl-N,3-diethyl-3H-purin-6-amine. In a preferred embodiment the PDE IV inhibitor is theophylline. In another preferred embodiment the PDE IV inhibitor is arofylline. In another preferred embodiment the PDE IV inhibitor is doxofylline. In another preferred embodiment the PDE IV inhibitor is dyphylline. In another preferred embodiment the PDE IV inhibitor is bamifylline. In another preferred embodiment the PDE IV inhibitor is ibudilast.

In another embodiment the PDE IV inhibitor is a benzofuran, benzopyran or related compound selected from the group consisting of lirimilast, (4-chlorophenyl)[3-(3,3-dihydroxybutyl)-6-hydroxy-1-benzofuran-2-yl]methanone, 1-{3-(dimethylamino)-4-[(dimethylamino)methyl]-7-hydroxy-5,6-dimethyl-1-benzofuran-2-yl}ethanone, N-(3,5-dichloropyridin-4-yl)-8-methoxy-2,2-dimethylchromane-5-carboxamide, and 2-acetyl-N-benzyl-7-methoxy-1-benzofuran-4-sulfonamide. In another embodiment the PDE IV inhibitor is selected from the group consisting of 1-cyclopentyl-N-(3,5-dichloropyridin-4-yl)-3-ethyl-1H-indazole-6-carboxamide, 1-cyclopentyl-3-ethyl-6-(2-methylphenyl)-1,3a,4,5,6,7a-hexahydro-7H-pyrazolo[3,4-c]pyridin-7-one, N-(4-oxo-1-phenyl-3,4,6,7-tetrahydro[1,4]diazepino[6,7,1-hi]indol-3-yl)-1H-indole-2-carboxamide, CI-1118, 4-[4-cyclopropyl-6-(cyclopropylamino)-1,3,5-triazin-2-yl]-llambda˜4˜,4-thiazinane-1,1-diol, N-cyclopropyl-4-(2-methylcyclopropyl)-6-(2-methylmorpholin-4-yl)-1,3,5-triazin-2-amine, and atizoram, filaminast, piclamilast, tibenelast, CDP 840, GW 3600, NCS 613, PDB 093, Ro 20-1724, RS 25344-000, SKF 107806, XT44, tolafentrine, zardaverine, T-2585, SDZ-ISQ-844, SB 207499, RPR-117658A, L-787258, E-4021, GF-248, IPL-4088, CP-353164, CP-146523, CP-293321, T-611,WAY-126120, WAY-122331, WAY-127093B, PDB-093, CDC-801, CC-7085, CDC-998, CH-3697, CH-3442, CH-2874, CH-4139, RPR-114597, RPR-122818, KF-19514, CH-422, CH-673, CH-928, KW-4490, Org 20241, Org 30029,VMX 554, VMX 565, benafentrine, trequinsin, EMD 54622, RS 17597, Nitraquazone, oxagrelate, T-440.

In the present invention the TNF alpha anagonist is an agent, compound, or molecule or a composition containing an agent, compound or molecule, including analogs, isomers, homologues, fragments or variants thereof, which antagonizes, inhibits, inactivates, reduces, suppresses, and/or limits the release, synthesis, or production from cells of TNF alpha.

Preferably the TNF-alpha antagonist is selected from the group consisting of a TNF-alpha antibody, a metalloproteinase inhibitor, a corticosteroid, a tetracycline TNF-alpha antagonist, a fluoroquinolone TNF-alpha antagonist, and a quinolone TNF-alpha antagonist.

In one embodiment the TNF-alpha antagonist is a TNF-alpha antibody. Preferably the TNF-alpha antibody is selected from the group consisting of infliximab, etanercept, CytoFAb, AGT-1, afelimomab, PassTNF, and CDP-870.

In another embodiment the TNF-alpha antagonist is a metalloproteinase inhibitor. Even more preferably the metalloproteinase inhibitor is a matrix metalloproteinase inhibitor.

In another embodiment the TNF-alpha antagonist is a corticosteroid. Preferably the corticosteroid is selected from the group consisting of mometasone, fluticasone, ciclesonide, budesonide, beclomethasone, beconase, flunisolide, deflazacort, betamethasone, methyl-prednisolone, dexamethasone, prednisolone, hydrocortisone, cortisol, triamcinolone, cortisone, corticosterone, dihydroxycortisone, beclomethasone dipropionate, and prednisone.

In another embodiment the TNF-alpha antagonist is a tetracycline TNF-alpha antagonist. Preferably the tetracycline TNF-alpha antagonist is selected from the group. consisting of doxycycline, minocycline, oxytetracycline, tetracycline, lymecycline, and 4-hydroxy-4-dimethylaminotetracycline.

In another embodiment the TNF-alpha antagonist is a fluoroquinolone TNF-alpha antagonist. Preferably the fluoroquinolone TNF-alpha antagonist is selected from the group consisting of norfloxacin, ofloxacin, ciprofloxacin, lomefloxacin, gatifloxacin, perfloxacin, and temafloxacin.

In another embodiment the TNF-alpha antagonist is a quinolone TNF-alpha antagonist. Preferably the quinolone TNF-alpha antagonist is selected from the group consisting of vesnarinone and amrinone.

In another embodiment the TNF-alpha antagonist is selected from the group consisting of thalidomide, Onercept, Pegsunercept, interferon-gamma, interleukin-1, pentoxyphylline, pimobeddan, lactoferrin, melatonin, nitrogen oxide, napthopyridine, a lazaroid, hydrazine sulfate, ketotifen, tenidap, a cyclosporin, peptide T, sulfasalazine, thorazine, an antioxidant, a cannabinoid, glycyrrhizin, sho-saiko-to, and L-camitine.

The present invention provides for a therapeutic composition for the treatment or prophylaxis of a PDE IV- or a TNF-alpha-related condition in a mammal in need of such treatment or prophylaxis comprising administrating to the mammal an amount of a PDE IV inhibitor and an amount of a TNF-alpha antagonist wherein the amount of the PDE IV inhibitor and the amount of the TNF-alpha antagonist together comprise an effective treatment or prevention of a PDE IV- or a TNF-alpha-related condition.

The therapeutic composition of the present invention comprises an amount of a PDE IV inhibitor and an amount of a TNF alpha antagonist.

The present invention also provides for a kit for the purpose of treatment or prophylaxis of a PDE IV- or a TNF-alpha-related condition in a mammal in need of such treatment or prophylaxis, the kit comprising a dosage form comprising a PDE IV inhibitor and a dosage form comprising a TNF-alpha antagonist.

Dosage Forms and Delivery System.

The PDE IV inhibitor, the TNF alpha antagonist, or pharmaceutical compositions comprising them may be administered enterally and parenterally. Oral (intra-gastric) is a preferred route of administration. The compounds useful in the present inventioncan be administered, for example, in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs. Topical dosage forms for administration of this invention include ointments, powders, sprays, inhalants, creams, jellies, collyriums, solutions or suspensions.

Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets can contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffm, or olive oil.

Aqueous suspensions can be produced that contain the active materials in admixture with excipients suitable for the manufacture of aqueous suspensions. Such excipients include suspending agents, for example sodium carboxymethylcellulose, methylcellulose, hydroxypropylmethyl-cellulose, sodium alginate, polyvinylpyrrolidone gum tragacanth and gum acacia. Dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. Another useful excipient is polyethylene oxide (PEG).

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the PDE IV inhibitor and/or the TNF alpha antagonist may be formulated with sweetening agents, for example glycerol, sorbitol, or sucrose. Such formulations may also contain a demulcent, a preservative and flavoring and coloring agents.

The subject method of prescribing a PDE IV inhibitor and a TNF alpha antagonist can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Also, administration can be delivered by inhalation, whether oral or nasal inhalation, according to the methods of the present invention can include formulations as are well known in the art, that are capable of being aerosolized for delivery by inhalation. A metered dose inhaler or a nebulizer provides aerosol delivery. Both devices are capable of providing delivery of a range of particle sizes including particles in the preferred range of about 1 micron to about 5 microns. Particles larger than about 10 microns are deposited primarily in the mouth and oropharynx, while particles smaller than about 0.5 microns are inhaled to the alveolae and then exhaled without significant deposition in the lungs. An alternative device for inhalant therapy is a dry powder inhaler using, for example, lactose or glucose powder to carry the therapeutic compound. For all forms of inhalant therapy, factors other than particle size and type of device also influence the amount of deposition in the lungs, including tidal volume, rate of breathing and breath holding. Therefore, an individual being instructed in inhalation therapy according to the methods of current invention should also be instructed to take slow deep breaths and hold each breath for several seconds, and preferably for about 5-10 seconds. Typically, the total daily dose of the therapeutic compounds according to the methods of the present invention will be administered as 1-4 puffs on a b.i.d-q.i.d. basis (i.e. twice-a-day, three times per day or four times a day), and as needed, or solely on an as-needed basis.

PDE IV Inhibitor Dosage Amount

Daily dosages can vary within wide limits and will be adjusted to the individual requirements in each particular case. In general, for administration to adults, an appropriate daily dosage has been described below, although the limits that were identified as being preferred may be exceeded if expedient. The daily dosage can be administered as a single dosage or in divided dosages. Various delivery systems include capsules, tablets, food, and gelatin capsules, for example.

TABLE 2

| PDE IV | Dosage | | |
|---|---|---|---|
| Inhibitor | Amount | | REFERENCE |
| Ariflo | 20-30 | mg per day | Souness, J., et al., Immunopharmacology, 47: 127-162 |
| | | | (2000) |
| Rolipram | 0.5-2 | mg/kg per day | Teixeira, M., et al., Memorias do Instituto Oswaldo Cruz, |
| | | | 92(II): 193-196 (1997); Souness, J., et al., |
| | | | Immunopharmacology, 47: 127-162 (2000) |
| Arofylline | 20 | mg per day | Souness, J., et al., Immunopharmacology, 47: 127-162 |
| | | | (2000) |
| Ibudilast | 40 | mg per day | Souness, J., et al., Immunopharmacology, 47: 127-162 |
| | | | (2000) |
| Tibenalast | 150 | mg per day | Souness, J., et al., Immunopharmacology, 47: 127-162 |
| | | | (2000) |
| Piclamilast | 0.2-0.8 | mg per day | Souness, J., et al., Immunopharmacology, 47: 127-162 |
| | | | (2000) |
| CDP-840 | 30 | mg per day | Souness, J., et al., Immunopharmacology, 47: 127-162 |
| | | | (2000) |
| RP 73401 | 2 | mg/kg per day | Teixeira, M., et al., Memorias do Instituto Oswaldo Cruz, |
| | | | 92(II): 193-196 (1997) |
| NVP- | 0.1-3 | mg/kg per day | Trifilieff, A., et al., J. Pharmacol. Exp. Ther., 301(1): |
| ABE171 | | | 241-248 (2002) |

The exact dosage and regimen for administering a PDE IV inhibitor will necessarily depend upon the potency and duration of action of the compounds used, the nature and severity of the illness to be treated, as well as the sex, age, weight, general health and individual responsiveness of the patient to be treated, and other relevant circumstances. While not intended to be limiting, an example of the normally prescribed dosage for the PDE IV inhibitor, roflumilast, has been reported to be about 0.5 mg once daily for human rhinitis treatment. See Schmidt, M. et al., J. Allergy Clin. Immunol. 108(4):530-536 (2001). In humans, roflumilast has been reported as effective when dosed at between about 0.01 and 0.5 mg/kg of body weight for inhalation and between about 0.05 and 2 mg/kg of body weight per day for systemic therapies. See U.S. Pat. No. 5,712,298.

Other examples of recommended PDE IV dosages are include in Table 2.

### Table 2

Therefore, for purposes of the present invention, it is preferred to dose the PDE IV inhibitor in an amount sufficient to provide a steroid-sparing benefit when given as a combination therapy to a subject in need of such treatment, wherein the amount of the PDE IV inhibitor which is administered and the amount of the corticosteroid which is administered together comprise a therapeutically effective amount of the combination.

More preferred is to dose the PDE IV inhibitor to a subject in need of such therapy between about 0.001 mg/kg and 10 mg/kg of body weight per day. More preferred, the PDE IV inhibitor should be dosed to the subject between about 0.01 and 5 mg/kg per day. Even more preferred still, the PDE IV inhibitor should be dosed to the subject between about 0.1 and 2.0 mg/kg per day.

TNF Alpha Antagonist Dosage Amount

Etanercept is known to those in the art. For adult patients the recommended dose of etanercept is 25 mg administered as a subcutaneous injection given twice a week at least 72-96 hours apart. Physician Desk Reference, 2002. For pediatric patients ages 4-17 years, the recommended dose of etanercept is 0.4/mg/kg (up to a maximum of 25 mg per dose) administered as a subcutaneous injection given twice a week at least 72-96 hours apart. Id.

Infliximab is know to those skilled in the art. The recommended dose of infliximab is 5 mg/kg administered as an intravenous infusion. Id. Infliximab is also administered in combination with methotrexzte. The recommended dose of infliximab in combination with methotrexate is 3 mg/kg administered as an intravenous infusion followed with additional similar doses at 2 and 6 weeks after the first infusion then every 8 weeks thereafter. Id.

Other examples of recommended TNF alpha antagonist dosages are include in Table 3.

TABLE 3

| TNF ALPHA | |
|---|---|
| ANTAGONIST | DOSAGE AND ROUTE OF ADMINISTRATION |
| Remicade (Inflixbimab) | Dose of 3 mg/kg given as an intravenous infusion |
| anti-tumor necrosis factor | followed w/ additional similar doses at 2 and 6 weeks |
| (TNF) monoclonal antibody | after the first infusion and then every 8 weeks thereafter |
| Embrel | 25 mg dose given twice weekly as a subcutaneous |
| (Etanercept) | injection 72-96 hours apart. |
| soluble TNF receptor fusion | |
| protein | |
| Methylprenisolone | 4-160 mg/day - suspension |
| Doxycycline | Oral & IV: 200 mg/day in adults on the first day, and |
| | thereafter 100 mg/day; 100 mg q 12 h for the entire |
| | course of therapy has also been used. In children 8 yr & |
| | older 4 mg/kg/day on the first day, and thereafter 2 mg/kg/day; |
| | 4 mg/kg/day for the entire course has also |
| | been used. |
| Minocycline | Oral & IV: 200 mg followed by 100 mg q 12 h in adults |
| | and in children 8 yrs & older 4 mg/kg followed by 2 mg/kg |
| | q 12 h. |
| Oxytetracycline | Oral: 250-500 mg q 6 h to adults and 25-50 mg/kg/day |
| | in children 8 yr & older. |
| | IV: 250-500 mg q 12 h to adults and 10-25 mg/kg/day |
| | in children 8 yr & older. |
| Tetracycline | Oral: 250-500 mg q 6 h to adults and 25-50 mg/kg/day |
| | in children 8 yr & older. |
| | IV: 250-500 mg q 12 h to adults and 10-25 mg/kg/day |
| | in children 8 yr & older. |
| Norfloxacin | Oral: 400 mg bid |
| Ofloxacin | Oral & IV: 200-400 mg bid |
| Ciprofloxacin | Oral: 250-750 mg bid |
| | IV: 200-400 mg q 12 h. |
| Gatifloxacin | Oral: 200 mg & 400 mg tablets |
| | IV: 20 mL (200 mg) & 40 mL (400 mg) single use vials |
| Amrinone | Loading dose: 40 mg IVP over 3 minutes (0.75 mg/kg) |
| | Maintenance dose: 250-900 mcg/min (5-10 mcg/kg/min) |
| Interferon-gamma | Interferon gamma 1b (Actimmune) injection 100 mcg |
| | (2 Million IU) |
| Thalidomide | Oral—100-400 mg per day |
| Pentoxyphylline | Oral- Controlled Release 400 mg tid |
| Melatonin | Oral - 3-10 mg per day |

Reference: Physicians' Desk Reference, 56^{th} Edition, 2002.

Therapeutic Uses

The present invention encompasses the therapeutic treatment of several inflammatory-related disorders. For example, the methods of the present invention are useful for the treatment of pulmonary inflammatory disorders, pulmonary hypertension, asthma, exercised induced asthma, pollution induced asthma, allergy induced asthma, COPD, osteoarthritis, adult respiratory distress syndrom, infant respiratory distress syndrom, retinitis, uveitis, glaucoma, retinopathy, diabetic angiopathy, edema formation, arthritis, rheumatoid arthritis, multiple sclerosis and Crohn's disease, chronic bronchitis, eosinophilic granuloma, psoriasis and other benign or malignant proliferative skin diseases, endotoxic shock (and associated conditions such as laminitis and colic in horses), septic shock, ulcerative colitis, reperfusion injury of the myocardium and brain, osteoporosis, chronic glomerulonephritis, atopic dermatitis, urticaria, adult respiratory distress syndrome, infant respiratory distress syndrome, chronic obstructive pulmonary disease, diabetes insipidus, rhinitis (including allergic rhinitis), allergic conjunctivitis, vernal conjunctivitis, arterial restenosis, atherosclerosis, neurogenic inflammation, pain, cough, ankylosing spondylitis, transplant rejection and graft versus host disease, hypersecretion of gastric acid, bacterial, fungal or viral induced sepsis or septic shock, inflammation and cytokine-mediated chronic tissue degeneration, cancer, cachexia, conjunctivitis, dermatitis, muscle wasting, depression, inflammatory bowel disease, allergic responses to insect and arthropod bites, memory impairment, monopolar depression, acute and chronic neurodegenerative disorders with inflammatory components, Parkinson disease, Alzheimer's disease, spinal cord trauma, head injury, joint injury, multiple sclerosis, tumor growth, and cancerous invasion of normal tissues, including any other disorders that are amenable to amelioration through inhibition of the PDE IV isoenzyme and the resulting elevated cAMP levels via administration to a patient in need of such treatment of an effective amount of the compounds referred to in the methods of the present invention.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

c. Assays and Screens

Inhibition of PDE Isoenzymes

The assay mixture contains 50 mM Tris (pH 7.4), 5 mM MgCl₂, 0.5 μM cAMP or cGMP, and [³H]cAMP or [³H]cGMP (about 30,000 cpm/assay), the indicated concentration of the inhibitor and an aliquot of the enzyme solution at a final assay volume of 200 μl.

Stock solutions of the compounds are diluted 1:100 (v/v) in the Tris buffer mentioned above; appropriate dilutions are prepared in 1% (v/v) DMSO/Tris buffer, which are diluted 1:2 (v/v) in the assays to obtain the desired fmal concentrations of the inhibitors at a DMSO concentration of 0.5% (v/v). DMSO itself affects none of the PDE activities.

After preincubation for 5 min at 37° C., the reaction isstarted by the addition of substrate (cAMP or cGMP) and the assays are incubated for further 15 min at 37° C. Then 50 μl of 0.2 N HCl is added to stop the reaction and the assays are left on ice for about 10 min. Following incubation with 25 μg of 5′-nucleotidase (Crotalus atrox snake venom) for 10 min at 37° C., the assays areloaded on QAE Sephadex A-25 (1 ml of bed volume in Poly-Prep chromatography columns; Bio-Rad, München, Germany). The columns are elutedwith 2 ml of 30 mM ammonium formate (pH 6.0) and the eluate is counted for radioactivity. Results are corrected for blank values (measured in the presence of denatured protein) that are below 5% of total radioactivity. The amount of cyclic nucleotides hydrolyzed does not exceed 30% of the original substrate concentration.

PDE1 from bovine brain is assayed in the presence of Ca²⁺ (1 mM) and calmodulin (100 nM) using cGMP as substrate. A blankvalue is measured in the presence of EGTA (1 mM) is subtracted from all values. PDE2 from rat heart is chromatographically purified and is assayed in the presence of cGMP (5 μM) using cAMP as substrate. PDE3 and PDE5 are assayed in the cytosol of human platelets using cAMP and cGMP, respectively, as substrate. PDE4 is tested in the cytosol of human neutrophils using cAMP as substrate. The PDE3-specific inhibitor motapizone (1 μM) is included to suppress PDE3 activity originating from contaminatingplatelets. See Hatzelmann, A., et al., J. Pharm. Exper. Therap., 297(1):267-279 (2001).

TNFα Assay

Cells areincubated in 96-well plates (Primaria 3872) at a density of 5×10⁴ cells/well in a total assay volume of 200 μl (RPMI 1640 medium containing 10% AB-serum for monocytes and macrophages, and Iscove's modified Dulbecco's medium containing 10% FBS for dendritic cells). Compounds (10 μl) are added 30 min before stimulation of the cells with “LPS working solution” (10 μl): a stock solution ofLPS (1 mg/ml, w/v) is prepared in 0.1% (v/v) hydroxylamine in PBS; after sonication for 5 min, 1-ml aliquots are stored at −20° C. Before starting the experiment, this solution is fiwther diluted in the corresponding cell-specific culture medium to get the LPS working solution. The appropriate cell-specific submaximal final LPS concentrations are determined in preliminary experiments and are 1 ng/ml for monocytes and 100 ng/ml for macrophages and dendritic cells. In the macrophage experiments, PGE₂ (10 nM) is added as a cAMP trigger to provideresponsiveness of the cells for PDE inhibitors.

Stock solutions of the compounds are diluted 1:50 (v/v) in medium; subsequent dilutions are made in 2% (v/v) DMSO/medium to achieve the final drug concentrations in the assays at a DMSO concentration of 0.1 % (v/v), which by itself does not affect TNFα synthesis. Starting from a 10 mM stock solution in DMSO, motapizone's further diluted in medium so that the resulting DMSO concentration at the final compound concentration (1 μM) could be neglected.

After overnight culture (about 13 h) in the case of monocytes and macrophages or 24 h in the case of dendritic cells, supernatants (about 180 μl) are removed and stored at −20° C. before TNFπ measurement by a commercially available enzymimmunoassay from Immunotech (Hamburg, Germany) performed essentially according to the manufacturer's instructions. See Hatzelmann, A., et al., J. Pharm. Exper. Therap., 297(1):267-279 (2001).

Lung Function/Capacity

The degree and severity of asthma and COPD can be determined by measuring lung expiratory flow volume and expiratory flow rates. Measurement can accomplished with, for example, a spirometer, flow volume loop, or pneumotach, before and after each of the treatments. Use of spirometry is a standard test for determining the efficacy of PDE IV inhibitors and corticosteroids after administration to a patient suffering from a pulmonary inflammatory disorder. A device called a spirometer is used to measure how much air the lungs can hold and how well the respiratory system is able to move air into and out of the lungs.

Spirometry is a medical test that measures the physical volume of air an individual forcibly inhales or exhales into a device. The objective of spirometry is to assess ventilatory function. An estimate of flow rate, or the rate at which the volume is changing as a function of time can also be calculated with spirometery. See SPIROMETRY The Measurement and Inteipretation of Ventilatory Function in Clinical Practice, Rob Pierce and David P. Johns, The Thoracic Society of Australia and New Zealand (1995). Thus, with the methods of the present invention, spirometric comparisons of pulmonary airflow before and after treatment will elucidate similarities and differences that enable one of skill to determine the effectiveness of the treatment methods.

Common parameters that spirometry measures are Forced Vital Capacity (FVC)—the maximum volume of air, measured in liters that can be forcibly and rapidly exhaled. Another parameter is Forced Expiratory Volume (FEV1)—the volume of air expelled in the first second of a forced expiration. Normal parameters for a patient not suffering from an inflammatory disorder such as asthma or COPD is: Tidal volume—5 to 7 milliliters per kilogram of body weight; Expiratory reserve volume—25% of vital capacity; Inspiratory capacity—75% of vital capacity forced expiratory volume—75% of vital capacity after 1 second, 94% after 2 seconds, and 97% after 3 seconds. Spirometry results are expressed as a percentage, and are considered abnormal if less than 80% of the normal predicted value. An abnormal result usually indicates the presence of some degree of obstructive lung disease such as COPD and chronic bronchitis, or restrictive lung disease such as pulmonary fibrosis or asthma.

### EXAMPLE 1.

table of Preferred Combinations

TABLE 4

| Example | | |
|---|---|---|
| Number | PDE IV Inhibitor | TNF alpha Inhibitor |
| 1 | arofylline & | Infliximab |
| 2 | arofylline & | Etanercept |
| 3 | arofylline & | CytoFAb |
| 4 | arofylline & | Afelimomab |
| 5 | arofylline & | PassTNF |
| 6 | arofylline & | CDP-870 |
| 7 | arofylline & | beclomethasone |
| 8 | arofylline & | beconase |
| 9 | arofylline & | budesonide |
| 10 | arofylline & | deflazacort |
| 11 | arofylline & | flunisolide |
| 12 | arofylline & | fluticasone |
| 13 | arofylline & | ketotifen |
| 14 | arofylline & | onercept |
| 15 | arofylline & | pentoxifylline |
| 16 | arofylline & | thalidomide |
| 17 | arofylline & | prednisone |
| 18 | arofylline & | triamcinolone |
| 19 | arofylline & | ciclesonide |
| 20 | arofylline & | Pegsunercept |
| 21 | atizoram & | Infliximab |
| 22 | atizoram & | Etanercept |
| 23 | atizoram & | CytoFAb |
| 24 | atizoram & | Afelimomab |
| 25 | atizoram & | PassTNF |
| 26 | atizoram & | CDP-870 |
| 27 | atizoram & | beclomethasone |
| 28 | atizoram & | beconase |
| 29 | atizoram & | budesonide |
| 30 | atizoram & | deflazacort |
| 31 | atizoram & | flunisolide |
| 32 | atizoram & | fluticasone |
| 33 | atizoram & | ketotifen |
| 34 | atizoram & | onercept |
| 35 | atizoram & | pentoxifylline |
| 36 | atizoram & | thalidomide |
| 37 | atizoram & | prednisone |
| 38 | atizoram & | triamcinolone |
| 39 | atizoram & | ciclesonide |
| 40 | atizoram & | Pegsunercept |
| 41 | AWD-12-281 & | Infliximab |
| 42 | AWD-12-281 & | Etanercept |
| 43 | AWD-12-281 & | CytoFAb |
| 44 | AWD-12-281 & | Afelimomab |
| 45 | AWD-12-281 & | PassTNF |
| 46 | AWD-12-281 & | CDP-870 |
| 47 | AWD-12-281 & | beclomethasone |
| 48 | AWD-12-281 & | beconase |
| 49 | AWD-12-281 & | budesonide |
| 50 | AWD-12-281 & | deflazacort |
| 51 | AWD-12-281 & | flunisolide |
| 52 | AWD-12-281 & | fluticasone |
| 53 | AWD-12-281 & | ketotifen |
| 54 | AWD-12-281 & | onercept |
| 55 | AWD-12-281 & | pentoxifylline |
| 56 | AWD-12-281 & | thalidomide |
| 57 | AWD-12-281 & | prednisone |
| 58 | AWD-12-281 & | triamcinolone |
| 59 | AWD-12-281 & | ciclesonide |
| 60 | AWD-12-281 & | Pegsunercept |
| 61 | bamifylline & | Infliximab |
| 62 | bamifylline & | Etanercept |
| 63 | bamifylline & | CytoFAb |
| 64 | bamifylline & | Afelimomab |
| 65 | bamifylline & | PassTNF |
| 66 | bamifylline & | CDP-870 |
| 67 | bamifylline & | beclomethasone |
| 68 | bamifylline & | beconase |
| 69 | bamifylline & | budesonide |
| 70 | bamifylline & | deflazacort |
| 71 | bamifylline & | flunisolide |
| 72 | bamifylline & | fluticasone |
| 73 | bamifylline & | ketotifen |
| 74 | bamifylline & | onercept |
| 75 | bamifylline & | pentoxifylline |
| 76 | bamifylline & | thalidomide |
| 77 | bamifylline & | prednisone |
| 78 | bamifylline & | triamcinolone |
| 79 | bamifylline & | ciclesonide |
| 80 | bamifylline & | Pegsunercept |
| 81 | CDC-801 & | Infliximab |
| 82 | CDC-801 & | Etanercept |
| 83 | CDC-801 & | CytoFAb |
| 84 | CDC-801 & | Afelimomab |
| 85 | CDC-801 & | PassTNF |
| 86 | CDC-801 & | CDP-870 |
| 87 | CDC-801 & | beclomethasone |
| 88 | CDC-801 & | beconase |
| 89 | CDC-801 & | budesonide |
| 90 | CDC-801 & | deflazacort |
| 91 | CDC-801 & | flunisolide |
| 92 | CDC-801 & | fluticasone |
| 93 | CDC-801 & | ketotifen |
| 94 | CDC-801 & | onercept |
| 95 | CDC-801 & | pentoxifylline |
| 96 | CDC-801 & | thalidomide |
| 97 | CDC-801 & | prednisone |
| 98 | CDC-801 & | triamcinolone |
| 99 | CDC-801 & | ciclesonide |
| 100 | CDC-801 & | Pegsunercept |
| 101 | CDP 840 & | Infliximab |
| 102 | CDP 840 & | Etanercept |
| 103 | CDP 840 & | CytoFAb |
| 104 | CDP 840 & | Afelimomab |
| 105 | CDP 840 & | PassTNF |
| 106 | CDP 840 & | CDP-870 |
| 107 | CDP 840 & | beclomethasone |
| 108 | CDP 840 & | beconase |
| 109 | CDP 840 & | budesonide |
| 110 | CDP 840 & | deflazacort |
| 111 | CDP 840 & | flunisolide |
| 112 | CDP 840 & | fluticasone |
| 113 | CDP 840 & | ketotifen |
| 114 | CDP 840 & | onercept |
| 115 | CDP 840 & | pentoxifylline |
| 116 | CDP 840 & | thalidomide |
| 117 | CDP 840 & | prednisone |
| 118 | CDP 840 & | triamcinolone |
| 119 | CDP 840 & | ciclesonide |
| 120 | CDP 840 & | Pegsunercept |
| 121 | cilomilast & | Infliximab |
| 122 | cilomilast & | Etanercept |
| 123 | cilomilast & | CytoFAb |
| 124 | cilomilast & | Afelimomab |
| 125 | cilomilast & | PassTNF |
| 126 | cilomilast & | CDP-870 |
| 127 | cilomilast & | beclomethasone |
| 128 | cilomilast & | beconase |
| 129 | cilomilast & | budesonide |
| 130 | cilomilast & | deflazacort |
| 131 | cilomilast & | flunisolide |
| 132 | cilomilast & | fluticasone |
| 133 | cilomilast & | ketotifen |
| 134 | cilomilast & | onercept |
| 135 | cilomilast & | pentoxifylline |
| 136 | cilomilast & | thalidomide |
| 137 | cilomilast & | prednisone |
| 138 | cilomilast & | triamcinolone |
| 139 | cilomilast & | ciclesonide |
| 140 | cilomilast & | Pegsunercept |
| 141 | cipamfylline & | Infliximab |
| 142 | cipamfylline & | Etanercept |
| 143 | cipamfylline & | CytoFAb |
| 144 | cipamfylline & | Afelimomab |
| 145 | cipamfylline & | PassTNF |
| 146 | cipamfylline & | CDP-870 |
| 147 | cipamfylline & | beclomethasone |
| 148 | cipamfylline & | beconase |
| 149 | cipamfylline & | budesonide |
| 150 | cipamfylline & | deflazacort |
| 151 | cipamfylline & | flunisolide |
| 152 | cipamfylline & | fluticasone |
| 153 | cipamfylline & | ketotifen |
| 154 | cipamfylline & | onercept |
| 155 | cipamfylline & | pentoxifylline |
| 156 | cipamfylline & | thalidomide |
| 157 | cipamfylline & | prednisone |
| 158 | cipamfylline & | triamcinolone |
| 159 | cipamfylline & | ciclesonide |
| 160 | cipamfylline & | Pegsunercept |
| 161 | D-4418 & | Infliximab |
| 162 | D-4418 & | Etanercept |
| 163 | D-4418 & | CytoFAb |
| 164 | D-4418 & | Afelimomab |
| 165 | D-4418 & | PassTNF |
| 166 | D-4418 & | CDP-870 |
| 167 | D-4418 & | beclomethasone |
| 168 | D-4418 & | beconase |
| 169 | D-4418 & | budesonide |
| 170 | D-4418 & | deflazacort |
| 171 | D-4418 & | flunisolide |
| 172 | D-4418 & | fluticasone |
| 173 | D-4418 & | ketotifen |
| 174 | D-4418 & | onercept |
| 175 | D-4418 & | pentoxifylline |
| 176 | D-4418 & | thalidomide |
| 177 | D-4418 & | prednisone |
| 178 | D-4418 & | triamcinolone |
| 179 | D-4418 & | ciclesonide |
| 180 | D-4418 & | Pegsunercept |
| 181 | doxofylline & | Infliximab |
| 182 | doxofylline & | Etanercept |
| 183 | doxofylline & | CytoFAb |
| 184 | doxofylline & | Afelimomab |
| 185 | doxofylline & | PassTNF |
| 186 | doxofylline & | CDP-870 |
| 187 | doxofylline & | beclomethasone |
| 188 | doxofylline & | beconase |
| 189 | doxofylline & | budesonide |
| 190 | doxofylline & | deflazacort |
| 191 | doxofylline & | flunisolide |
| 192 | doxofylline & | fluticasone |
| 193 | doxofylline & | ketotifen |
| 194 | doxofylline & | onercept |
| 195 | doxofylline & | pentoxifylline |
| 196 | doxofylline & | thalidomide |
| 197 | doxofylline & | prednisone |
| 198 | doxofylline & | triamcinolone |
| 199 | doxofylline & | ciclesonide |
| 200 | doxofylline & | Pegsunercept |
| 201 | dyphylline & | Infliximab |
| 202 | dyphylline & | Etanercept |
| 203 | dyphylline & | CytoFAb |
| 204 | dyphylline & | Afelimomab |
| 205 | dyphylline & | PassTNF |
| 206 | dyphylline & | CDP-870 |
| 207 | dyphylline & | beclomethasone |
| 208 | dyphylline & | beconase |
| 209 | dyphylline & | budesonide |
| 210 | dyphylline & | deflazacort |
| 211 | dyphylline & | flunisolide |
| 212 | dyphylline & | fluticasone |
| 213 | dyphylline & | ketotifen |
| 214 | dyphylline & | onercept |
| 215 | dyphylline & | pentoxifylline |
| 216 | dyphylline & | thalidomide |
| 217 | dyphylline & | prednisone |
| 218 | dyphylline & | triamcinolone |
| 219 | dyphylline & | ciclesonide |
| 220 | dyphylline & | Pegsunercept |
| 221 | ibudilast & | Infliximab |
| 222 | ibudilast & | Etanercept |
| 223 | ibudilast & | CytoFAb |
| 224 | ibudilast & | Afelimomab |
| 225 | ibudilast & | PassTNF |
| 226 | ibudilast & | CDP-870 |
| 227 | ibudilast & | beclomethasone |
| 228 | ibudilast & | beconase |
| 229 | ibudilast & | budesonide |
| 230 | ibudilast & | deflazacort |
| 231 | ibudilast & | flunisolide |
| 232 | ibudilast & | fluticasone |
| 233 | ibudilast & | ketotifen |
| 234 | ibudilast & | onercept |
| 235 | ibudilast & | pentoxifylline |
| 236 | ibudilast & | thalidomide |
| 237 | ibudilast & | prednisone |
| 238 | ibudilast & | triamcinolone |
| 239 | ibudilast & | ciclesonide |
| 240 | ibudilast & | Pegsunercept |
| 241 | KW 4490 & | Infliximab |
| 242 | KW 4490 & | Etanercept |
| 243 | KW 4490 & | CytoFAb |
| 244 | KW 4490 & | Afelimomab |
| 245 | KW 4490 & | PassTNF |
| 246 | KW 4490 & | CDP-870 |
| 247 | KW 4490 & | beclomethasone |
| 248 | KW 4490 & | beconase |
| 249 | KW 4490 & | budesonide |
| 250 | KW 4490 & | deflazacort |
| 251 | KW 4490 & | flunisolide |
| 252 | KW 4490 & | fluticasone |
| 253 | KW 4490 & | ketotifen |
| 254 | KW 4490 & | onercept |
| 255 | KW 4490 & | pentoxifylline |
| 256 | KW 4490 & | thalidomide |
| 257 | KW 4490 & | prednisone |
| 258 | KW 4490 & | triamcinolone |
| 259 | KW 4490 & | ciclesonide |
| 260 | KW 4490 & | Pegsunercept |
| 261 | L-791943 & | Infliximab |
| 262 | L-791943 & | Etanercept |
| 263 | L-791943 & | CytoFAb |
| 264 | L-791943 & | Afelimomab |
| 265 | L-791943 & | PassTNF |
| 266 | L-791943 & | CDP-870 |
| 267 | L-791943 & | beclomethasone |
| 268 | L-791943 & | beconase |
| 269 | L-791943 & | budesonide |
| 270 | L-791943 & | deflazacort |
| 271 | L-791943 & | flunisolide |
| 272 | L-791943 & | fluticasone |
| 273 | L-791943 & | ketotifen |
| 274 | L-791943 & | onercept |
| 275 | L-791943 & | pentoxifylline |
| 276 | L-791943 & | thalidomide |
| 277 | L-791943 & | prednisone |
| 278 | L-791943 & | triamcinolone |
| 279 | L-791943 & | ciclesonide |
| 280 | L-791943 & | Pegsunercept |
| 281 | lirimilast & | Infliximab |
| 282 | lirimilast & | Etanercept |
| 283 | lirimilast & | CytoFAb |
| 284 | lirimilast & | Afelimomab |
| 285 | lirimilast & | PassTNF |
| 286 | lirimilast & | CDP-870 |
| 287 | lirimilast & | beclomethasone |
| 288 | lirimilast & | beconase |
| 289 | lirimilast & | budesonide |
| 290 | lirimilast & | deflazacort |
| 291 | lirimilast & | flunisolide |
| 292 | lirimilast & | fluticasone |
| 293 | lirimilast & | ketotifen |
| 294 | lirimilast & | onercept |
| 295 | lirimilast & | pentoxifylline |
| 296 | lirimilast & | thalidomide |
| 297 | lirimilast & | prednisone |
| 298 | lirimilast & | triamcinolone |
| 299 | lirimilast & | ciclesonide |
| 300 | lirimilast & | Pegsunercept |
| 301 | ONO-6126 & | Infliximab |
| 302 | ONO-6126 & | Etanercept |
| 303 | ONO-6126 & | CytoFAb |
| 304 | ONO-6126 & | Afelimomab |
| 305 | ONO-6126 & | PassTNF |
| 306 | ONO-6126 & | CDP-870 |
| 307 | ONO-6126 & | beclomethasone |
| 308 | ONO-6126 & | beconase |
| 309 | ONO-6126 & | budesonide |
| 310 | ONO-6126 & | deflazacort |
| 311 | ONO-6126 & | flunisolide |
| 312 | ONO-6126 & | fluticasone |
| 313 | ONO-6126 & | ketotifen |
| 314 | ONO-6126 & | onercept |
| 315 | ONO-6126 & | pentoxifylline |
| 316 | ONO-6126 & | thalidomide |
| 317 | ONO-6126 & | prednisone |
| 318 | ONO-6126 & | triamcinolone |
| 319 | ONO-6126 & | ciclesonide |
| 320 | ONO-6126 & | Pegsunercept |
| 321 | PD-189659 & | Infliximab |
| 322 | PD-189659 & | Etanercept |
| 323 | PD-189659 & | CytoFAb |
| 324 | PD-189659 & | Afelimomab |
| 325 | PD-189659 & | PassTNF |
| 326 | PD-189659 & | CDP-870 |
| 327 | PD-189659 & | beclomethasone |
| 328 | PD-189659 & | beconase |
| 329 | PD-189659 & | budesonide |
| 330 | PD-189659 & | deflazacort |
| 331 | PD-189659 & | flunisolide |
| 332 | PD-189659 & | fluticasone |
| 333 | PD-189659 & | ketotifen |
| 334 | PD-189659 & | onercept |
| 335 | PD-189659 & | pentoxifylline |
| 336 | PD-189659 & | thalidomide |
| 337 | PD-189659 & | prednisone |
| 338 | PD-189659 & | triamcinolone |
| 339 | PD-189659 & | ciclesonide |
| 340 | PD-189659 & | Pegsunercept |
| 341 | pentoxifylline & | Infliximab |
| 342 | pentoxifylline & | Etanercept |
| 343 | pentoxifylline & | CytoFAb |
| 344 | pentoxifylline & | Afelimomab |
| 345 | pentoxifylline & | PassTNF |
| 346 | pentoxifylline & | CDP-870 |
| 347 | pentoxifylline & | beclomethasone |
| 348 | pentoxifylline & | beconase |
| 349 | pentoxifylline & | budesonide |
| 350 | pentoxifylline & | deflazacort |
| 351 | pentoxifylline & | flunisolide |
| 352 | pentoxifylline & | fluticasone |
| 353 | pentoxifylline & | ketotifen |
| 354 | pentoxifylline & | onercept |
| 355 | pentoxifylline & | thalidomide |
| 356 | pentoxifylline & | prednisone |
| 357 | pentoxifylline & | triamcinolone |
| 358 | pentoxifylline & | ciclesonide |
| 359 | pentoxifylline & | Pegsunercept |
| 360 | piclamilast & | Infliximab |
| 361 | piclamilast & | Etanercept |
| 362 | piclamilast & | CytoFAb |
| 363 | piclamilast & | Afelimomab |
| 364 | piclamilast & | PassTNF |
| 365 | piclamilast & | CDP-870 |
| 366 | piclamilast & | beclomethasone |
| 367 | piclamilast & | beconase |
| 368 | piclamilast & | budesonide |
| 369 | piclamilast & | deflazacort |
| 370 | piclamilast & | flunisolide |
| 371 | piclamilast & | fluticasone |
| 372 | piclamilast & | ketotifen |
| 373 | piclamilast & | onercept |
| 374 | piclamilast & | pentoxifylline |
| 375 | piclamilast & | thalidomide |
| 376 | piclamilast & | prednisone |
| 377 | piclamilast & | triamcinolone |
| 378 | piclamilast & | ciclesonide |
| 379 | piclamilast & | Pegsunercept |
| 380 | pumafentrin & | Infliximab |
| 381 | pumafentrin & | Etanercept |
| 382 | pumafentrin & | CytoFAb |
| 383 | pumafentrin & | Afelimomab |
| 384 | pumafentrin & | PassTNF |
| 385 | pumafentrin & | CDP-870 |
| 386 | pumafentrin & | beclomethasone |
| 387 | pumafentrin & | beconase |
| 388 | pumafentrin & | budesonide |
| 389 | pumafentrin & | deflazacort |
| 390 | pumafentrin & | flunisolide |
| 391 | pumafentrin & | fluticasone |
| 392 | pumafentrin & | ketotifen |
| 393 | pumafentrin & | onercept |
| 394 | pumafentrin & | pentoxifylline |
| 395 | pumafentrin & | thalidomide |
| 396 | pumafentrin & | prednisone |
| 397 | pumafentrin & | triamcinolone |
| 398 | pumafentrin & | ciclesonide |
| 399 | pumafentrin & | Pegsunercept |
| 400 | roflumilast & | Infliximab |
| 401 | roflumilast & | Etanercept |
| 402 | roflumilast & | CytoFAb |
| 403 | roflumilast & | Afelimomab |
| 404 | roflumilast & | PassTNF |
| 405 | roflumilast & | CDP-870 |
| 406 | roflumilast & | beclomethasone |
| 407 | roflumilast & | beconase |
| 408 | roflumilast & | budesonide |
| 409 | roflumilast & | deflazacort |
| 410 | roflumilast & | flunisolide |
| 411 | roflumilast & | fluticasone |
| 412 | roflumilast & | ketotifen |
| 413 | roflumilast & | onercept |
| 414 | roflumilast & | pentoxifylline |
| 415 | roflumilast & | thalidomide |
| 416 | roflumilast & | prednisone |
| 417 | roflumilast & | triamcinolone |
| 418 | roflumilast & | ciclesonide |
| 419 | roflumilast & | Pegsunercept |
| 420 | rolipram & | Infliximab |
| 421 | rolipram & | Etanercept |
| 422 | rolipram & | CytoFAb |
| 423 | rolipram & | Afelimomab |
| 424 | rolipram & | PassTNF |
| 425 | rolipram & | CDP-870 |
| 426 | rolipram & | beclomethasone |
| 427 | rolipram & | beconase |
| 428 | rolipram & | budesonide |
| 429 | rolipram & | deflazacort |
| 430 | rolipram & | flunisolide |
| 431 | rolipram & | fluticasone |
| 432 | rolipram & | ketotifen |
| 433 | rolipram & | onercept |
| 434 | rolipram & | pentoxifylline |
| 435 | rolipram & | thalidomide |
| 436 | rolipram & | prednisone |
| 437 | rolipram & | triamcinolone |
| 438 | rolipram & | ciclesonide |
| 439 | rolipram & | Pegsunercept |
| 440 | SCH-351591 & | Infliximab |
| 441 | SCH-351591 & | Etanercept |
| 442 | SCH-351591 & | CytoFAb |
| 443 | SCH-351591 & | Afelimomab |
| 444 | SCH-351591 & | PassTNF |
| 445 | SCH-351591 & | CDP-870 |
| 446 | SCH-351591 & | beclomethasone |
| 447 | SCH-351591 & | beconase |
| 448 | SCH-351591 & | budesonide |
| 449 | SCH-351591 & | deflazacort |
| 450 | SCH-351591 & | flunisolide |
| 451 | SCH-351591 & | fluticasone |
| 452 | SCH-351591 & | ketotifen |
| 453 | SCH-351591 & | onercept |
| 454 | SCH-351591 & | pentoxifylline |
| 455 | SCH-351591 & | thalidomide |
| 456 | SCH-351591 & | prednisone |
| 457 | SCH-351591 & | triamcinolone |
| 458 | SCH-351591 & | ciclesonide |
| 459 | SCH-351591 & | Pegsunercept |
| 460 | T-440 & | Infliximab |
| 461 | T-440 & | Etanercept |
| 462 | T-440 & | CytoFAb |
| 463 | T-440 & | Afelimomab |
| 464 | T-440 & | PassTNF |
| 465 | T-440 & | CDP-870 |
| 466 | T-440 & | beclomethasone |
| 467 | T-440 & | beconase |
| 468 | T-440 & | budesonide |
| 469 | T-440 & | deflazacort |
| 470 | T-440 & | flunisolide |
| 471 | T-440 & | fluticasone |
| 472 | T-440 & | ketotifen |
| 473 | T-440 & | onercept |
| 474 | T-440 & | pentoxifylline |
| 475 | T-440 & | thalidomide |
| 476 | T-440 & | prednisone |
| 477 | T-440 & | triamcinolone |
| 478 | T-440 & | ciclesonide |
| 479 | T-440 & | Pegsunercept |
| 480 | Theophylline & | Infliximab |
| 481 | Theophylline & | Etanercept |
| 482 | Theophylline & | CytoFAb |
| 483 | Theophylline & | Afelimomab |
| 484 | Theophylline & | PassTNF |
| 485 | Theophylline & | CDP-870 |
| 486 | Theophylline & | beclomethasone |
| 487 | Theophylline & | beconase |
| 488 | Theophylline & | budesonide |
| 489 | Theophylline & | deflazacort |
| 490 | Theophylline & | flunisolide |
| 491 | Theophylline & | fluticasone |
| 492 | Theophylline & | ketotifen |
| 493 | Theophylline & | onercept |
| 494 | Theophylline & | pentoxifylline |
| 495 | Theophylline & | thalidomide |
| 496 | Theophylline & | prednisone |
| 497 | Theophylline & | triamcinolone |
| 498 | Theophylline & | ciclesonide |
| 499 | Theophylline & | Pegsunercept |
| 500 | V-11294A & | Infliximab |
| 501 | V-11294A & | Etanercept |
| 502 | V-11294A & | CytoFAb |
| 503 | V-11294A & | Afelimomab |
| 504 | V-11294A & | PassTNF |
| 505 | V-11294A & | CDP-870 |
| 506 | V-11294A & | beclomethasone |
| 507 | V-11294A & | beconase |
| 508 | V-11294A & | budesonide |
| 509 | V-11294A & | deflazacort |
| 510 | V-11294A & | flunisolide |
| 511 | V-11294A & | fluticasone |
| 512 | V-11294A & | ketotifen |
| 513 | V-11294A & | onercept |
| 514 | V-11294A & | pentoxifylline |
| 515 | V-11294A & | thalidomide |
| 516 | V-11294A & | prednisone |
| 517 | V-11294A & | triamcinolone |
| 518 | V-11294A & | ciclesonide |
| 519 | V-11294A & | Pegsunercept |
| 520 | YM-976 & | Infliximab |
| 521 | YM-976 & | Etanercept |
| 522 | YM-976 & | CytoFAb |
| 523 | YM-976 & | Afelimomab |
| 524 | YM-976 & | PassTNF |
| 525 | YM-976 & | CDP-870 |
| 526 | YM-976 & | beclomethasone |
| 527 | YM-976 & | beconase |
| 528 | YM-976 & | budesonide |
| 529 | YM-976 & | deflazacort |
| 530 | YM-976 & | flunisolide |
| 531 | YM-976 & | fluticasone |
| 532 | YM-976 & | ketotifen |
| 533 | YM-976 & | onercept |
| 534 | YM-976 & | pentoxifylline |
| 535 | YM-976 & | thalidomide |
| 536 | YM-976 & | prednisone |
| 537 | YM-976 & | triamcinolone |
| 538 | YM-976 & | ciclesonide |
| 539 | YM-976 & | Pegsunercept |

The inventionbeing thus described, it is apparent that the same can be varied in many ways. Such variations are not to be regarded as a departure from the spirit and scope of the present invention, and all such modifications and equivalents as would be obvious to one skilled in the art are intended to be included within the scope of the following clims.

What is claimed is:

1. A method for the treatment or prophylaxis of a PDE IV- or a TNF-alpha-related condition in a mammal in need of such treatment or prophylaxis comprising administrating to the mammal an amount of a PDE IV inhibitor and an amount of a TNF-alpha antagonist wherein the amount of the PDE IV inhibitor and the amount of the TNF-alpha antagonist together comprise a therapy effective for the treatment or prophylaxis of a PDE IV- or a TNF-alpha-related condition.

2. The method of claim 1, wherein the TNF-alpha antagonist is selected from the group consisting of a metalloproteinase inhibitor, a tetracycline TNF-alpha antagonist, a fluoroquinolone TNF-alpha antagonist, and a quinolone TNF-alpha antagonist.

3. The method of claim 1, wherein the PDE IV inhibitor is selected from the group consisting of roflumilast, cilomilast, ZK-117137, bamifylline, dyphylline, ibudilast, and theophylline.

4. The method of claim 1, wherein the PDE IV inhibitor is selected from the group consisting of a quinazolinedione PDE IV inhibitor, a xanthine PDE IV inhibitor, and a benzamide PDE IV inhibitor.

5. The method of claim 4, wherein the PDE IV inhibitor is selected from the group consisting of 1-cyclopentyl-N-(3,5-dichloropyridin-4-yl)-3-ethyl-1H-indazole-6-carboxamide, 1-cyclopentyl-3-ethyl-6-(2-methylphenyl)-1,3a,4,5,6,7a-hexahydro-7H-pyrazolo[3,4-c]pyridin-7-one, N-(4-oxo-1-phenyl-3,4,6,7-tetrahydro[1,4]diazepino[6,7,1-hi]indol-3-yl)-1H-indole-2-carboxamide, CI-1118, 4-[4-cyclopropyl-6-(cyclopropylamino)-1,3,5-triazin-2-yl]-llambda˜4˜,4-thiazinane-1,1-diol, and N-cyclopropyl-4-(2-methylcyclopropyl)-6-(2-methylmorpholin-4-yl)-1,3,5-triazin-2-amine, atizoram, filaminast, piclamilast, tibenelast, CDP 840, GW 3600, NCS 613, PDB 093, Ro 20-1724, RS 25344-000, SKF 107806, XT-44, tolafentrine, zardaverine,T-2585, SDZ-ISQ-844, SB 207499, RPR-117658A, L-787258, E-4021, GF-248, IPL-4088, CP-353164, CP-146523, CP-293321, T-611,WAY-126120, WAY-122331,WAY-127093B, PDB-093, CDC-801, CC-7085, CDC-998, CH-3697, CH-3442, CH-2874, CH-4139, RPR-114597, RPR-122818, KF-19514, CH-422, CH-673, CH-928, KW-4490, Org 20241, Org 30029,VMX 554, VMX 565, benafentrine, trequinsin, EMD 54622, RS 17597, Nitraquazone, oxagrelate, T-440.

6. The method of claim 2, wherein the TNF-alpha antagonist is a TNF-alpha antibody.

7. The method of claim 6, wherein the TNF-alpha antibody is selected from the group consisting of infliximab, etanercept, CytoFAb, AGT-1, afelimomab, PassTNF, and CDP-870.

8. The method of claim 2, wherein the TNF-alpha antagonist is selected from the group consisting of thalidomide, Onercept, Pegsunercept, interferon-gamma, interleukin-1, pentoxyphylline, pimobeddan, lactoferrin, melatonin, nitrogen oxide, napthopyridine, a lazaroid, hydrazine sulfate, ketotifen, tenidap, a cyclosporin, peptide T, sulfasalazine, thorazine, an antioxidant, a cannabinoid, glycyrrhizin, sho-saiko-to, and L-camitine.

9. A therapeutic composition comprising an amount of a PDE IV inhibitor and an amount of a TNF-alpha antagonist and a pharmaceutically acceptable excipient.

10. The therapeutic composition of claim 9, wherein the PDE IV inhibitor is selected from the group consisting of roflumilast, cilomilast, ZK- 117137, bamifylline, dyphylline, ibudilast, and theophylline.

11. The therapeutic composition of claim 9, wherein the PDE IV inhibitor is selected from the group consisting of a catechol ether PDE IV inhibitor, a quinazolinedione PDE IV inhibitor, a xanthine PDE IV inhibitor, and a benzamide PDE IV inhibitor.

12. The therapeutic composition of claim 11, wherein the PDE IV inhibitor is selected from the group consisting of 1-cyclopentyl-N-(3,5-dichloropyridin-4-yl)-3-ethyl-1H-indazole-6-carboxamide, 1 -cyclopentyl-3-ethyl-6-(2-methylphenyl)-1,3a,4,5,6,7a-hexahydro-7H-pyrazolo[3,4-c]pyridin-7-one, N-(4-oxo-1-phenyl-3,4,6,7-tetrahydro[1,4]diazepino[6,7,1-hi]indol-3-yl)-1H-indole-2-carboxamide, CI-1118, 4-[4-cyclopropyl-6-(cyclopropylamino)-1,3,5-triazin-2-yl]-llambda˜4˜,4-thiazinane-1,1-diol, and N-cyclopropyl-4-(2-methylcyclopropyl)-6-(2-methylmorpholin-4-yl)-1,3,5-triazin-2-amine, atizoram, filaminast, piclamilast, tibenelast, CDP 840, GW 3600, NCS 613, PDB 093, Ro 20-1724, RS 25344-000, SKF 107806, XT-44, tolafentrine, zardaverine, T-2585, SDZ-ISQ-844, SB 207499, RPR-117658A, L-787258, E-4021, GF-248, IPL-4088, CP-353164, CP-146523, CP-293321, T-611,WAY-126120, WAY-122331,WAY-127093B, PDB-093, CDC-801, CC-7085, CDC-998, CH-3697, CH-3442, CH-2874, CH-4139, RPR-114597, RPR-122818, KF-19514, CH422, CH-673, CH-928, KW-4490, Org 20241, Org 30029,VMX 554, VMX 565, benafentrine, trequinsin, EMD 54622, RS 17597, Nitraquazone, oxagrelate, T-440.

13. The therapeutic composition of claim 9, wherein the TNF-alpha antagonist is a TNF-alpha antibody.

14. The therapeutic composition of claim 13, wherein the TNF-alpha antibody is selected from the group consisting of infliximab, etanercept, CytoFAb, AGT-1, afelimomab, PassTNF, and CDP-870.

15. A kit for the purpose of treatment or prophylaxis of a PDE IV- or a TNF-alpha-related condition in a mammal in need of such treatment or prophylaxis, the kit comprising a dosage form comprising a PDE IV inhibitor and a dosage form comprising a TNF-alpha antagonist.
